(19) United States
(12) Patent Application Publication
(10) Pub. No.: US 2004/0127435 A1
(43) Pub. Date: Jul. 1, 2004

(54) Uses for inhibitors of inosine monophosphate dehydrogenase

(75) Inventors: Dennis A. Carson, Del Mar, CA (US); Lorenzo M. Leoni, San Diego, CA (US); Howard B. Cottam, Escondido, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: REGENTS OF THE UNIVERSITY OF CALIFORNIA, Oakland, CA

(21) Appl. No.: 10/632,711

(22) Filed: Aug. 1, 2003

Related U.S. Application Data
(60) Provisional application No. 60/400,583, filed on Aug. 2, 2002.

(51) Int. Cl. A61K 31/7056; A61K 31/4745; A61K 31/337

(52) U.S. Cl. 514/43; 514/283; 514/575; 514/449; 514/629

(57) ABSTRACT

The present invention provides methods of treating cancer using inhibitors of inosine monophosphate dehydrogenase (IMPDH). The IMPDH inhibitors are combined with compounds that inhibit cellular processes regulated by GTP or ATP. Also provided are prodrugs of the IMPDH inhibitor mizoribine and its aglycone. The prodrugs are useful in practicing the methods of the invention, including immunosuppressive therapy and treatment of cancer by prolonged administration without additional therapeutic compounds.

### CROSS-REFERENCES TO RELATED APPLICATIONS

The present application claims the benefit of U.S. Provisional Application No. 60/400,583, filed Aug. 2, 2002, which is herein incorporated by reference in its entirety.

The present application is related to U.S. Provisional Application No. 60/400,568, filed Aug. 2, 2002 and to U.S. Ser. No. ______, Attorney Docket No. 02307O-126810US, filed Aug. 1, 2003, both of which are herein incorporated by reference in their entirety.

### STATEMENT AS TO RIGHTS TO INVENTIONS MADE UNDER FEDERALLY SPONSORED RESEARCH AND DEVELOPMENT

This invention was made with Government support under Grant No. GM 23200, awarded by the National Institutes of Health. The Government has certain rights in this invention.

### BACKGROUND OF THE INVENTION

Inosine monophosphate dehydrogenase (IMPDH) is a key enzyme in the synthesis of guanine nucleotides. The enzyme is a rate-limiting enzyme in de novo GTP biosynthesis. See e.g., Yalowitz and Jayaram, Cancer Research, 20:2319-2338 (2000), catalyzing the dehydrogenation of IMP to xanthosine 5′-monophosphate (XMP).

Nucleotides are required for cells to divide and to replicate and IMPDH activity is upregulated in some cancer cells. Thus, inhibitors of IMPDH are attractive candidates for targeting diseases characterized by unregulated cell division, e.g., cancer. Inhibitors of IMPDH are known, however, these inhibitors are not always selective. See, e.g. WO 00/26197. Thus, the effectiveness of known IMPDH inhibitors as chemotherapeutic agents is limited.

The present invention solves this and other problems.

### BRIEF SUMMARY OF THE INVENTION

In one aspect, this invention provides a method of treating cancer, comprising administering to a subject a therapeutically effective amount of a combination of compounds: an IMPDH inhibitor, or an enantiomer, prodrug or a pharmaceutically acceptable salt of an IMPDH inhibitor, combined with another drug, preferably a drug that affects a cellular process regulated by GTP or ATP levels. The combination of compounds provides synergistic, beneficial results for cancer treatment. The invention also provides compositions including an IMPDH inhibitor, or an enantiomer, prodrug or a pharmaceutically acceptable salt of an IMPDH inhibitor, combined with another drug, preferably a drug that affects a cellular process regulated by GTP or ATP levels. The invention also provides novel combinations of IMPDH inhibitors with other compounds, described in detail below.

In some embodiments cancer is treated by administration of an inhibitor of inosine monophosphate dehydrogenase (IMPDH), or enantiomer, prodrug or a pharmaceutically acceptable salt of an IMPDH inhibitor; in combination with an inhibitor of α-tubulin polymerization, or a prodrug or pharmaceutically acceptable salt of an inhibitor of α-tubulin polymerization. IMPDH inhibitors include mizoribine, mizoribine aglycone, mycophenolate mofetil, tiazofurin, viramidine, and ribivarin. A-tubulin polymerization inhibitors include indanocine, indanorine, vincristine, vinblastine, vinorelbine, combretastatin-A, and colchicine.

In one embodiment, the cancer is treated by administration of the IMPDH inhibitor mizoribine in combination with the α-tubulin polymerization inhibitors indanocine or indanorine. In another embodiment, a population of cells comprising the cancer that is treated by administration of this combination is shown to have a high rate of α-tubulin turnover.

In some embodiments a slow growing cancer is treated with the combination of an IMPDH inhibitor and a α-tubulin polymerization inhibitor, or an enantiomer, prodrug or a pharmaceutically acceptable salt of an IMPDH inhibitor and/or a α-tubulin polymerization inhibitor. Slow growing cancers include chronic lymphocytic leukemia (CLL), chronic myelogenous leukemia, non-Hodgkins lymphoma, multiple myeloma, chronic granulocytic leukemia, cutaneous T cell lymphoma, low grade lymphomas, slowly proliferating breast cancer, slowly proliferating prostate cancer, and slowly proliferating thyroid cancer. In a preferred embodiment, the slow growing cancer is CLL.

The invention also provides compositions including an inhibitor of IMPDH, or an enantiomer, prodrug, or a pharmaceutically acceptable salt of an inhibitor of IMPDH; in combination with an inhibitor of α-tubulin polymerization, or a prodrug or pharmaceutically acceptable salt of an inhibitor of α-tubulin polymerization. IMPDH inhibitors include mizoribine, mizoribine aglycone, mycophenolate mofetil, tiazoftirin, viramidine, and ribivarin. A-tubulin polymerization inhibitors include indanocine, indanorine, vincristine, vinblastine, vinorelbine, combretastatin-A, and colchicine. In a preferred embodiment, the composition includes the IMPDH inhibitor mizoribine and the α-tubulin polymerization inhibitor indanocine.

In another embodiment of the invention, cancer is treated by administering a combination of an inhibitor of IMPDH, or an enantiomer, or a prodrug, or a pharmaceutically acceptable salt of an IMPDH inhibitor; and a precursor of 9-beta-D-arabinofuranosylguanine 5′-triphosphate (Ara-GTP), or a prodrug, or a pharmaceutically acceptable salt of a precursor of Ara-GTP. IMPDH inhibitors include mizoribine, mizoribine aglycone, mycophenolate mofetil, tiazofirin, viramidine, and ribivarin. Ara-GTP precursors include guanine arabinoside (Ara-G) and Nelarabine. In some embodiments, a lymphoma or a leukemia is treated with the combination of IMPDH inhibitor and an Ara-GTP precursor.

The invention also encompasses compositions of an inhibitor of IMPDH, or an enantiomer, or a prodrug, or a pharmaceutically acceptable salt an IMPDH inhibitor and a precursor of 9-beta-D-arabinofuranosylguanine 5′-triphosphate (Ara-GTP), or a prodrug, or a pharmaceutically acceptable salt of a precursor of Ara-GTP. IMPDH inhibitors include mizoribine, mizoribine aglycone, mycophenolate mofetil, tiazofurin, viramidine, and ribivarin. Ara-GTP precursors include guanine arabinoside (Ara-G) and Nelarabine.

In another embodiment of the invention, a cancer deficient in the enzyme methlyadenosine phosphorylase (MTAP) is treated by administering an inhibitor of IMPDH, or an enantiomer, or a prodrug, or a pharmaceutically acceptable salt an IMPDH inhibitor. IMPDH inhibitors include mizoribine, mizoribine aglycone, mycophenolate mofetil, tiazofurin, viramidine, and ribivarin. In a preferred embodiment, the IMPDH inhibitor is mizoribine or mizoribine aglycone.

In another embodiment of the invention, a cancer is treated by administering the IMPDH inhibitor in combination with an inhibitor of the de novo pathway of purine biosynthesis, or a prodrug or pharmaceutically acceptable salt of an inhibitor of purine biosynthesis. In a preferred embodiment, the inhibitor of the de novo pathway of purine biosynthesis inhibits adenylate succinate synthase (ASS), such as does L-alanosine or an antifolate. This combination of an IMPDH inhibitor and an inhibitor of the de novo pathway of purine biosynthesis can be used to treat cancer that is deficient in MTAP activity or a cancer that has apparently normal MTAP activity.

The invention also encompasses compositions of inhibitors of IMPDH, or an enantiomer, or a prodrug, or a pharmaceutically acceptable salt of an IMPDH inhibitor combined with an inhibitor of the de novo pathway of purine biosynthesis. In a preferred embodiment, the inhibitor of the de novo pathway of purine biosynthesis is L-alanosine or an antifolate. Preferred antifolates include methotrexate, trimetrexate, pemetrexed, 10-propargyl-5,8-dideazafolic acid (PDDF), N-[5-[N-(3,4-dihydro-2-methyl-4-oxoquinazolin-6-ylmethyl)-N-methylamino]-2-thenoyl]-L-glutamic acid (ZD 1694, Tomudex), N-[4-[2-(2-amino-3,4-dihydro-4-oxo-7H-pyrrolo[2,3-d]-pyrimidin-5-yl)ethyl]-benzoyl]-L-glutamic acid (LY231514), 6-(2′-formyl-2′naphthyl-ethyl)-2-amino-4(3H1)-oxoquinazoline (LL95 509), (6R, S)-5, 10-dideazatetrahydrofolic acid (DDATHF), 4-[2-(2-amino-4-oxo-4,6,7,8-tetrahydro-3Hpyrimidino[5,4,6][1,4]-thiazin-6yl)-(S)-ethyl]-2,5-thienoylamino-L-glutamic acid (AG2034), and N-[5-(2-[(2,6-diamino-4(3H)-oxopyrimidin-5-yl)thio]ethyl)thieno-2-yl]-L-glutamic acid (AG2009). Enantiomers, or a prodrugs, or a pharmaceutically acceptable salts of an inhibitor of the de novo pathway of purine biosynthesis can also be combined with an IMPDH inhibitor.

In a further embodiment, a cancer is treated with a combination of an inhibitor of a receptor tyrosine kinase, a prodrug therefor, or a pharmaceutically acceptable salt thereof; and an IMPDH inhibitor. The IMPDH inhibitor can be selected from mizoribine, mizoribine aglycone, mycophenolate mofetil, tiazofurin, viramidine, and ribivarin; and also includes enantiomers, prodrugs and pharmaceutically acceptable salts of those compounds. The receptor tyrosine kinase inhibitor can be selected from the group consisting of ST1571 (Gleevec), ZD1839 (Iressa), OSI-774, PKI116, GW2016, EKB-569, and CI1033, as well as enantiomers, prodrugs and pharmaceutically acceptable salts of those compounds. While the combination of IMPDH inhibitors and inhibitors of receptor tyrosine kinases can be used to treat many different cancers, in preferred embodiments, the treated cancers include gastrointestinal stromal tumor, non-small-cell lung cancer, squamous cell carcinoma of the head and neck, and hormone refractory prostate cancer.

The invention also encompasses compositions of inhibitors of IMPDH, or an enantiomer, or a prodrug, or a pharmaceutically acceptable salt of an IMPDH inhibitor combined with an inhibitor of a receptor tyrosine kinase. In a preferred embodiment, the inhibitor of a receptor tyrosine kinase is ST1571 (Gleevec), ZD1839 (Iressa), OSI-774, PKI116, GW2016, EKB-569, or CI1033, or an enantiomer, prodrug and pharmaceutically acceptable salt of those compounds.

In a further embodiment, a cancer is treated with a combination of an antagonist of a G-protein coupled receptor (GPCR), a prodrug therefor, or a pharmaceutically acceptable salt thereof; and an IMPDH inhibitor. The IMPDH inhibitor can be selected from mizoribine, mizoribine aglycone, mycophenolate mofetil, tiazofurin, viramidine, and ribivarin; and also includes enantiomers, prodrugs and pharmaceutically acceptable salts of those compounds. The antagonist of a GPCR can be selected from the group consisting of atrasentan, leuprolide, goserelin, and octreotide, as well as enantiomers, prodrugs and pharmaceutically acceptable salts of those compounds. While the combinations of an IMPDH inhibitor and an antagonist of a GPCR can be used to treat many different cancers, in preferred embodiments, the treated cancers include prostate cancer.

The invention also encompasses compositions of inhibitors of IMPDH, or an enantiomer, or a prodrug, or a pharmaceutically acceptable salt of an IMPDH inhibitor combined with an antagonist of a GPCR. In a preferred embodiment, the antagonist of a GPCR is atrasentan, leuprolide, goserelin, or octreotide, or an enantiomer, prodrug and pharmaceutically acceptable salt of those compounds.

Research has also focused on the development of new therapeutic agents which are in the form of prodrugs, compounds that are capable of being converted to drugs (active therapeutic compounds) in vivo by certain chemical or enzymatic modifications of their structure. For purposes of reducing toxicity, this conversion is preferably confined to the site of action or target tissue rather than the circulatory system or non-target tissue. However, even prodrugs are problematic as many are characterized by a low stability in blood and serum, due to the presence of enzymes that degrade or activate the prodrugs before the prodrugs reach the desired sites within the patient's body.

Thus, in another aspect, the present invention provides prodrugs of the IMPDH inhibitor mizoribine, its aglycone and its analogues. The invention provides compounds having the formula:

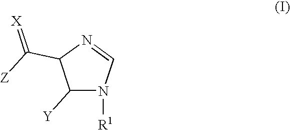

wherein, the symbol R¹ represents H, substituted or unsubstituted alkyl, substituted or 30 unsubstituted heteroalkyl or saccharyl moieties. The symbol X represents O, S or NR², in which R² is a member selected from H, substituted or unsubstituted alkyl, substituted or unsubstituted heteroalkyl, OH and NH². The symbol Y represents OR³ or NHR³, in which R³ is a member selected from H, substituted or unsubstituted alkyl, substituted or unsubstituted heteroalkyl, acyl and P(O)OR¹²R¹³. R¹² and R¹³ are members independently selected from H, substituted or unsubstituted alkyl, substituted or unsubstituted heteroalkyl, acyl, acyloxyalkyl, and a single bond to an oxygen of the saccharyl of R¹. The symbol Z represents NR⁴R⁵, OR⁴ and SR⁴, in which R⁴ represents H, substituted or unsubstituted alkyl, substituted or unsubstituted heteroalkyl, a single bond to R³ or acyl; and R⁵ represents H, substituted or unsubstituted alkyl, substituted or unsubstituted heteroalkyl, acyl, acyloxycarbonyl, amino acid, peptidyl or acyloxyalkyl moieties.

In another embodiment of the invention, a cancer is treated with mizoribine, mizoribine aglycone, prodrugs of mizoribine, or prodrugs of mizoribine aglycone by administration for a prolonged period of time. In one embodiment, the administration is carried out so that the plasma level of mizoribine, mizoribine aglycone, prodrugs of mizoribine, or prodrugs of mizoribine aglycone is between 0.5 and 50 micromolar for between 6 and 72 hours. In another embodiment, the administration is carried out so that the plasma level of mizoribine, mizoribine aglycone, prodrugs of mizoribine, or prodrugs of mizoribine aglycone is between 1 and 30 micromolar for between 8 and 48 hours. In a further embodiment, the administration is carried out so that the plasma level of mizoribine, mizoribine aglycone, prodrugs of mizoribine, or prodrugs of mizoribine aglycone is between 5 and 25 micromolar for between 10 and 24 hours. In a preferred embodiment, the plasma level of mizoribine, mizoribine aglycone, prodrugs of mizoribine, or prodrugs of mizoribine aglycone is at least 10 micromolar for at least 12 hours.

In a further embodiment, the mizoribine, mizoribine aglycone, prodrugs of mizoribine, or prodrugs of mizoribine aglycone includes a pharmaceutically acceptable carrier. The mizoribine, mizoribine aglycone, prodrugs of mizoribine, or prodrugs of mizoribine aglycone can be administered parenterally or orally.

In another embodiment, a prodrug of mizoribine or mizoribine aglycone used in any of the foregoing methods of treatment or compositions is described by the formula shown above.

In another embodiment of the invention, an immune system condition is treated by providing an immunosupressive agent, e.g., a therapeutically effective amount of a prodrug of mizoribine or mizoribine aglycone, as described above by Formula I. In a preferred embodiment, the compound includes a pharmaceutical carrier. The immune system condition can be rejection of a transplanted organ or an autoimmune disease, or other immune system conditions in which treatment of a subject with an immunosuppressive agent provides a beneficial effect. The IMPDH inhibitory compounds of Formula I can be used in the methods and compositions described in this disclosure, e.g., combinations of IMPDH inhibitors, including prodrugs, with an agent that inhibits a cellular process that is regulated by ATP or GTP. In addition, the compounds of Formula I are also useful to treat conditions that are treated by EIPDH inhibitors alone.

Other aspects, objects and advantages of the invention will be apparent from the detailed description that follows.

### BRIEF DESCRIPTION OF THE DRAWINGS

FIG. 1 depicts α-α-tubulin turnover in peripheral blood mononuclear cells (PBL) and chronic lymphocytic leukemia (CLL) cells. The cytosolic (S) monomeric and particulate-bound (P) polymerized forms of α-tubulin were separated by centrifugation from drug-treated cells and assayed quantitatively by immunoblotting with a specific monoclonal antibody. In normal PBL (left panel), α-tubulin was found mostly in the soluble fraction, with an apparent molecular weight of 61 kDa. Treatment with the microtubule-polymerizing agent paclitaxel for 1 hour did not change this pattern. PBL activated for 24 hours in presence of anti-CD3 and anti-CD28 antibodies (middle panel) displayed the majority of α-tubulin in the soluble fraction, with an apparent molecular weight of 54 kDa (lower band). However, activated PBL treated with paclitaxel displayed a shift of α-tubulin to the particulate-bound fraction. CLL cells (right panel) expressed almost exclusively the 54 kDa α-tubulin band in the soluble subcellular fraction. Treatment with paclitaxel induced the complete relocalization of α-tubulin to the particulate fraction.

FIG. 2 depicts the synergistic effect of treatment of CLL cells with mizoribine, an IMPDH inhibitor prodrug, and indanocine (depicted as 178), an α-tubulin polymerization inhibitor.

FIG. 3 depicts the effect of mizoribine treatment on MTAP-deleted chronic myelogenous leukemia cells. MTAP-deleted chronic myelogenous leukemia cells (K562) were pre-treated for the indicated times (24, 48, 72 hours) with concentrations of mizoribine (squares) or mizoribine base (triangles) from 200 μM to 0.5 μM. Cell proliferation was tested by the MTT assay at the end of the incubation time.

FIG. 4 depicts the effect treating MTAP-deleted chronic myelogenous leukemia cells with a combination of mizoribine-base and L-alanosine. MTAP-deleted lung cancer cells (A549) were pre-treated for 24 hours with control vehicle (square), or the indicated concentrations of mizoribine-base (10 μM, 25 μM and 50 μM). After 24 hours in culture L-alanosine was added at decreasing concentrations (½ dilutions) starting at 40 μM for an additional 48 hours of incubation. The proliferation of the cells was tested by the MTT assay.

### DETAILED DESCRIPTION OF THE INVENTION

Introduction

The present invention is generally directed to compositions and methods for the treatment of cancers, using novel combinations of IMPDH inhibitors and other compounds. In addition, the invention provides methods of prolonged administration of mizoribine and prodrugs of mizoribine, either alone or in combination with other compounds, to effectively treat cancer. The invention also provides novel mizoribine and mizoribine aglycone prodrugs for use in cancer and immunosuppressive therapy.

Inosine 5′-monophosphate dehydrogenase (IMPDH) is a rate-limiting enzyme in GTP biosynthesis. Inhibition of IMPDH activity, thus, can lead to a decrease in levels of GTP. Metabolism of GTP, in turn, is required for and regulates many essential cell processes including DNA synthesis, microtubule assembly and disassembly, cellular responses to G-protein coupled receptors, and intracellular signaling by G-proteins. In addition, because levels of GTP and ATP rise and fall in tandem due to equilibration by the enzyme nucleoside diphosphate synthase, inhibition of IMPDH activity also can lead to a decrease in the level of ATP and affect cellular processes regulated by cellular levels of ATP.

The combination of IMPDH inhibitors with other compounds that affect cellular processes regulated by GTP and/or ATP, provides enhanced toxicity to cancer cells, as compared to the toxicity that could be expected when either agent is administered to cancer cells alone. For example, microtubule dynamics and α-tubulin polymerization are affected by the ratio of GMP to GTP. Increased levels of GTP with respect to GMP leads to enhanced α-tubulin polymerization and microtubule formation, which cells need to divide, grow, and move. Conversely, a decrease in the GTP:GMP ratio shifts the dynamics in favor α-tubulin depolymerization and disassembly of microtubules. Thus, IMPDH inhibitors are combined with compounds that inhibit microtubule assembly and α-tubulin polymerization to potentiate the effect of such compounds. Cancer cells are thus inhibited by the effects of decreased GTP levels caused by the IMPDH inhibitor, as well as by the inhibitory effects on growth and/or movement caused by the compound that inhibits microtubule assembly and α-tubulin polymerization.

IMPDH inhibitors can also be combined with Ara-G and related compounds to treat cancer. Ara-G is a precursor of Ara-GTP, which is a purine analog that incorporates into DNA and, thus, terminates DNA synthesis. When cellular GTP levels are lowered by administration of an IMPDH inhibitor, cells in need of GTP will more readily take up the purine analog, incorporate it into DNA and thereby inhibit DNA synthesis and associated cellular processes, e.g., cell division, growth, and/or motility. The inhibitory actions of the combined treatment is greater than the inhibitory action of either compound acting alone.

Some cancer cells are deficient in de novo metabolism of purines and, as a result, increase their uptake of adenine, adenosine or their analogs. Some IMPDH inhibitors, such as mizoribine aglycone, are adenosine or adenine analogs and, thus, can be used alone or in combination with other compounds to treat cancer cells with deficient de novo metabolism of purines. Such IMPDH inhibitors more readily enter the cell and thus, exert a greater effect than if administered to a cancer cell that is not deficient in de novo metabolism of purines. IMPDH inhibitors that are not adenine or adenosine analogs can also be administered to cancer cells in combination with compounds that reduce de novo purine synthesis. The combination of IMPDH inhibitors with inhibitors of de novo purine biosynthesis can be used to treat cancer cells deficient in purine metabolism or to treat cancer cells with apparently normal purine metabolism.

Because IMPDH inhibitors affect the levels of nucleosides including ATP, IMPDH inhibitors can also be combined with inhibitors of tyrosine kinases to treat cancer.

IMPDH inhibitors and prodrugs disclosed in this application can be used alone to treat cancer. In a preferred embodiment the IMPDH inhibitors or prodrugs are administered to a patient so that plasma levels of the compounds are relatively high for a prolonged period of time.

Definitions

As used herein, “cancer” includes solid tumors and hematological malignancies. The former includes cancers such as breast, colon, and ovarian cancers. The latter include hematopoietic malignancies including leukemias, lymphomas and myelomas. This invention provides new effective methods, compositions, and kits for treatment and/or prevention of various types of cancer.

Hematological malignancies, such as leukemias and lymphomas, are conditions characterized by abnormal growth and maturation of hematopoietic cells.

Leukemias are generally neoplastic disorders of hematopoietic stem cells, and include adult and pediatric acute myeloid leukemias (AML), chronic myeloid leukemia (CML), acute lymphocytic leukemia (ALL), chronic lymphocytic leukemia (CLL), hairy cell leukemia and secondary leukemia. Myeloid leukemias are characterized by infiltration of the blood, bone marrow, and other tissues by neoplastic cells of the hematopoietic system. CLL is characterized by the accumulation of mature-appearing lymphocytes in the peripheral blood and is associated with infiltration of bone marrow, the spleen and lymph nodes.

Specific leukemias include acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, promyelocytic leukemia, Rieder cell leukemia, Schilling's leukemia, stem cell leukemia, subleukemic leukemia, and undifferentiated cell leukemia.

Lymphomas are generally neoplastic transformations of cells that reside primarily in lymphoid tissue. Among lymphomas, there are two major distinct groups: non-Hodgkin's lymphoma (NHL) and Hodgkin's disease. Lymphomas are tumors of the immune system and generally are present as both T cell- and as B cell-associated disease. Bone marrow, lymph nodes, spleen and circulating cells are all typically involved. Treatment protocols include removal of bone marrow from the patient and purging it of tumor cells, often using antibodies directed against antigens present on the tumor cell type, followed by storage. The patient is then given a toxic dose of radiation or chemotherapy and the purged bone marrow is then reinfused in order to repopulate the patient's hematopoietic system.

Other hematological malignancies include myelodysplastic syndromes (MDS), myeloproliferative syndromes (MPS) and myelomas, such as solitary myeloma and multiple myeloma. Multiple myeloma (also called plasma cell myeloma) involves the skeletal system and is characterized by multiple tumorous masses of neoplastic plasma cells scattered throughout that system. It may also spread to lymph nodes and other sites such as the skin. Solitary myeloma involves solitary lesions that tend to occur in the same locations as multiple myeloma.

Hematological malignancies are generally serious disorders, resulting in a variety of symptoms, including bone marrow failure and organ failure. Treatment for many hematological malignancies, including leukemias and lymphomas, remains difficult, and existing therapies are not universally effective. While treatments involving specific immunotherapy appear to have considerable potential, such treatments have been limited by the small number of known malignancy-associated antigens. Moreover the ability to detect such hematological malignancies in their early stages can be quite difficult depending upon the particular malady. Accordingly, there remains a need in the art for improved methods for treatment of hematological malignancies such as B cell leukemias and lymphomas and multiple myelomas. The present invention fulfills these and other needs in the field.

Other cancers are also of concern, and represent similar difficulties insofar as effective treatment is concerned. Such cancers include those characterized by solid tumors. Examples of other cancers of concern are skin cancers, including melanomas, basal cell carcinomas, and squamous cell carcinomas. Epithelial carcinomas of the head and neck are also encompassed by the present invention. These cancers typically arise from mucosal surfaces of the head and neck and include salivary gland tumors.

The present invention also encompasses cancers of the lung. Lung cancers include squamous or epidermoid carcinoma, small cell carcinoma, adenocarcinoma, and large cell carcinoma. Breast cancer is also included, both invasive breast cancer and non-invasive breast cancer, e.g., ductal carcinoma in situ and lobular neoplasia.

The present invention also encompasses gastrointestinal tract cancers. Gastrointestinal tract cancers include esophageal cancers, gastric adenocarcinoma, primary gastric lymphoma, colorectal cancer, small bowel tumors and cancers of the anus. Pancreatic cancer and cancers that affect the liver are also of concern, including hepatocellular cancer. The present invention also includes treatment of bladder cancer and renal cell carcinoma.

The present invention also encompasses prostatic carcinoma and testicular cancer.

Gynecologic malignancies are also encompassed by the present invention including ovarian cancer, carcinoma of the fallopian tube, uterine cancer, and cervical cancer.

Treatment of sarcomas of the bone and soft tissue are encompassed by the present invention. Bone sarcomas include osteosarcoma, chondrosarcoma, and Ewing's sarcoma.

The present invention also encompasses malignant tumors of the thyroid, including papillary, follicular, and anaplastic carcinomas.

As used herein a “slow growing cancer” is a cancer that is present in a subject in need of treatment, wherein the subject has more than a 50% survival rate after 5 years, even if at the time of diagnosis, the cancer has spread to the regional lymph nodes. See, e.g., Greenlee, R. T., et al., CA Cancer J. Clin. 50:7-33 (2000). In addition, slow growing cancers can include the following: chronic lymphocytic leukemia (CLL), chronic myelogenous leukemia, non-Hodgkins lymphoma, multiple myeloma, chronic granulocytic leukemia, cutaneous T cell lymphoma, low grade lymphomas, colon cancer, uterine cancer, breast cancer, prostate cancer, and thyroid cancer.

A “subject in need of treatment” is a mammal with cancer that is life-threatening or that impairs health or shortens the lifespan of the mammal.

A “pharmaceutically acceptable” component is one that is suitable for use with humans and/or animals without undue adverse side effects (such as toxicity, irritation, and allergic response) commensurate with a reasonable benefit/risk ratio.

A “safe and effective amount” refers to the quantity of a component that is sufficient to yield a desired therapeutic response without undue adverse side effects (such as toxicity, irritation, or allergic response) commensurate with a reasonable benefit/risk ratio when used in the manner of this invention. By “therapeutically effective amount” is meant an amount of a component effective to yield the desired therapeutic response, for example, an amount effective to delay the growth of a cancer or to cause a cancer to shrink or not metastasize. The specific safe and effective amount or therapeutically effective amount will vary with such factors as the particular condition being treated, the physical condition of the patient, the type of mammal being treated, the duration of the treatment, the nature of concurrent therapy (if any), and the specific formulations employed and the structure of the compounds or its derivatives.

A “pharmaceutically acceptable carrier” is a carrier, such as a solvent, suspending agent or vehicle, for delivering the compound or compounds in question to the animal or human. The carrier may be liquid or solid and is selected with the planned manner of administration in mind. Liposomes are also a pharmaceutical carrier. As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Carriers for use in the compositions of this invention are described in more detail below

As used herein, “IMPDH” is inosine 5′-monophosphate dehydrogenase, a rate-limiting enzyme in de novo GTP biosynthesis. An “IMPDH inhibitor” is a compound that reduces the activity of the enzyme. In some embodiments, an “IMPDH inhibitor” is a compound that reduces the activity of the enzyme by binding to the enzyme. Thus, an “IMPDH inhibitor” can inhibit activity of the enzyme in a competitive, or a noncompetitive manner.

As used herein “α-tubulin polymerization” is a GTP-dependent process where α-tubulin dimers are assembled into multimeric structures, including microtubules. An “α-tubulin polymerization inhibitor” is a compound that inhibits the polymerization of α-tubulin into multimeric structures. In a preferred embodiment, an “α-tubulin polymerization inhibitor” is a compound that interacts with α-tubulin at or near the GTP binding site. An α-tubulin polymerization inhibitor also encompasses compounds that interact with the vinca alkaloid or colchicine binding sites on α-tubulin.

As used herein, “a cellular process regulated by GTP” is a process that responds to GTP levels. Such processes include DNA and RNA synthesis, α-tubulin polymerization and depolymerization, and purine biosynthesis. Response can occur through regulation of enzymatic activity of the process, e.g., an enzyme is activated or inhibited in response to cellular GTP levels or the cellular ratio of GTP to GDP or GMP, for example. Regulation or response can also occur if GTP is a substrate or product for an enzyme in the cellular process. An “agent that inhibits a cellular process regulated by GTP” is a compound that detectably reduces or even halts the cellular process.

As used herein, “a cellular process regulated by ATP” is a process that responds to ATP levels. Such processes include DNA and RNA synthesis, kinase activity, phosphatase activity, and purine biosynthesis. Response can occur through regulation of enzymatic activity of the process, e.g., an enzyme is activated or inhibited in response to cellular ATP levels or the cellular ratio of ATP to ADP or AMP, for example. Regulation or response can also occur if ATP is a substrate or product for an enzyme in the cellular process. An “agent that inhibits a cellular process regulated by ATP” is a compound that detectably reduces or even halts the cellular process.

As used herein, “ara-GTP” is a purine analog that is incorporated into DNA, terminating DNA synthesis. See e.g., Gandhi, Hematology 1999 463-469. A “precursor of ara-GTP” is a compound that is given to a subject and then converted to the active form of Ara-GTP through the action of one or more enzymes. Precursors of Ara-GTP include Ara-G and Nelarabine, as well as other 6-substituted beta-D-arabinofuranosylpurines that are converted to guanosine analogs by either adenosine deaminase or xanthine oxidase.

As used herein, “tyrosine kinase” refers to an enzyme that phosphorylates a tyrosine residue on a protein using ATP as a substrate. Examples of tyrosine kinases include Bcr-Abl, Abl, PDGFR, c-kit and members of the epidermal growth factor receptor family. A “tyrosine kinase inhibitor” is a compound that specifically inhibits the activity of a tyrosine kinase. Examples of tyrosine kinase inhibitors include Gleevec, also known as imatinab mesylate or STI571; Iressa, a quinazoline also known as ZD1839; OSI-774; PKI 116; GW2016; EKB-569; and CI1033, also known as PD183805.

As used herein, an “antifolate” is a compound that inhibits an enzyme involved in synthesis of tetrahydrofolate or an intracellular tetrahydrofolate derivative. Tetrahydrofolate and its derivatives are important donors of one-carbon units during metabolism. Thus, antifolates also inhibit enzymes that use tetrahydrofolate or its derivatives as cofactors and sources of single carbon units. Enzymes that are inhibited by antifolates include dihydrofolate reductase, folylpolyglutamate synthetase (FPGS), glycinamide ribonucleotide formyltransferase (GARFT), and aminoimidazolecarboxamide ribonucleotide formyltransferase (AICARFT). Exemplary antifolates include methotrexate, trimetrexate, pemetrexed, 10-propargyl-5,8-dideazafolic acid (PDDF), N-[5-[N-(3,4-dihydro-2-methyl-4-oxoquinazolin-6-ylmethyl)-N-methylamino]-2-thenoyl]-L-glutamic acid (ZD1694, Tomudex), N-[4-[2-(2-amino-3,4-dihydro-4-oxo-7H-pyrrolo[2,3-d]-pyrimidin-5-yl)ethyl]-benzoyl]-L-glutamic acid (LY231514), 6-(2 ′-formyl-2′naphthyl-ethyl)-2-amino-4(3H)-oxoquinazoline (LL95509), (6R,S)-5,10-dideazatetrahydrofolic acid (DDATHF), 4-[2-(2-amino-4-oxo-4,6,7,8-tetrahydro-3Hpyrimidino[5,4,6][1,4]-thiazin-6yl)-(S)-ethyl]-2,5-thienoylamino-L-glutamic acid (AG2034), and N-[5-(2-[(2,6-diamino-4(3H)-oxopyrimidin-5-yl)thio]ethyl)thieno-2-yl]-L-glutamic acid (AG2009).

As used herein, “de novo pathway of purine biosynthesis” refers to enzymatic synthesis of purine in a multi-step pathway beginning with the formation of phosphoribosyl pyrophosphate (PRPP) and continuing to the synthesis of inosine monophosphate (IMP). The de novo pathway of purine biosynthesis also includes synthesis of precursors or cofactors of the substituents of the pathway, e.g., folate, tetrahydrofolate and derivatives. The de novo pathway of purine biosynthesis also includes enzymatic reactions that synthesize AMP, GMP, and corresponding diphosphates and triphosphates.

As used herein, “an immune system condition” is a condition in which an immune response is pathogenic or harmful to a patient. Rejection of a transplanted organ is one example of an immune system condition. Transplanted organs can include kidney, liver, heart, pancreas, bone marrow and heart-lung transplants. Other examples of immune system conditions include contact dermatitis; graft-vs-host disease in which donor immunological cells present in the graft attack host tissues in the recipient of the graft; diseases with proven or possible autoimmune components (e.g., an autoimmune disease), such as rheumatoid arthritis, psoriasis, autoimmune uveitis, multiple sclerosis, allergic encephalomyelitis, systemic lupus erythematosus, aplastic anemia, pure red cell anemia, idiopathic thrombocytopenia, scleroderma, chronic active hepatitis, myasthenia gravis, Crohn's disease, ulcerative colitis, Graves ophthalmopathy, sarcoidosis, primary biliary cirrhosis, primary juvenile diabetes, uveitis posterior, and interstitial lung fibrosis.

As used herein, “an immunosuppressive agent” is a drug or substance that suppresses an immune response. Exemplary immunosuppressive agents include mizoribine and mizoribine aglycone and analogues of same described in this application.

The symbol

[symbol]

whether utilized as a bond or displayed perpendicular to a bond indicates the point at which the displayed moiety is attached to the remainder of the molecule, solid support, etc.

The term “alkyl,” by itself or as part of another substituent, means, unless otherwise stated, a straight or branched chain, or cyclic hydrocarbon radical, or combination thereof, which may be fully saturated, mono- or polyunsaturated and can include di- and multivalent radicals, having the number of carbon atoms designated (i.e. C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkyl,” unless otherwise noted, is also meant to include those derivatives of alkyl defined in more detail below, such as “heteroalkyl.” Alkyl groups, which are limited to hydrocarbon groups are termed “homoalkyl”.

The term “alkylene” by itself or as part of another substituent means a divalent radical derived from an alkane, as exemplified, but not limited, by —CH₂CH₂CH₂CH₂—, and further includes those groups described below as “heteroalkylene.” Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms.

The term “heteroalkyl,” by itself or in combination with another term, means, unless otherwise stated, a stable straight or branched chain, or cyclic hydrocarbon radical, or combinations thereof, consisting of the stated number of carbon atoms and at least one heteroatom selected from the group consisting of O, N, Si and S, and wherein the nitrogen, carbon and sulfur atoms may optionally be oxidized and the nitrogen heteroatom may optionally be quaternized. The heteroatom(s) O, N and S and Si may be placed at any interior position of the heteroalkyl group or at the position at which the alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂,—S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. Up to two heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). The terms “heteroalkyl” and “heteroalkylene” encompass poly(ethylene glycol) and its derivatives (see, for example, Shearwater Polymers Catalog, 2001). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—.

The term “lower” in combination with the terms “alkyl” or “heteroalkyl” refers to a moiety having from 1 to 6 carbon atoms.

acyl (e.g., —C(O)CH₃, —C(O)CF₃, —C(O)CH₂OCH₃, and the like)

The terms “cycloalkyl” and “heterocycloalkyl”, by themselves or in combination with other terms, represent, unless otherwise stated, cyclic versions of substituted or unsubstituted “alkyl” and substituted or unsubstituted “heteroalkyl”, respectively. Additionally, for heterocycloalkyl, a heteroatom can occupy the position at which the heterocycle is attached to the remainder of the molecule. Examples of cycloalkyl include, Dut are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. The heteroatoms and carbon atoms of the cyclic structures are optionally oxidized.

Substituents for the alkyl, and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) are generally referred to as “alkyl substituents” and “heteroalkyl substituents,” respectively, and they can be one or more of a variety of groups selected from, but not limited to: —OR′, ═O, ═NR′, =N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂ in a number ranging from zero to (2m′+1), where m′ is the total number of carbon atoms in such radical. R′, R″, R′″ and R″″ each preferably independently refer to hydrogen, substituted or unsubstituted heteroalkyl, substituted or unsubstituted aryl, e.g., aryl substituted with 1-3 halogens, substituted or unsubstituted alkyl, alkoxy or thioalkoxy groups, or arylalkyl groups. When a compound of the invention includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

The term “saccharyl,” refers to substituents that are derived from a saccharide. The saccharide is without limitation a mono-, oligo, or poly-saccharide. The saccharyl moiety may be derived from a natural saccharide, an unnatural saccharide or a saccharide that is structurally modified by chemical or enzymatic methods. The remainder of the molecule of the invention is attached to the saccharyl moiety at any oxygen position of the sugar.

As used herein, the term “acyloxyalkyl,” refers to the group —C(O)O—R.

The term “acyloxycarbonyl” refers to the group —C(O)OC(O)—.

As used herein, the term “heteroatom” includes oxygen (O), nitrogen (N), sulfur (S) and silicon (Si).

The symbol “R” is a general abbreviation that represents a substituent group that is selected from substituted or unsubstituted alkyl, substituted or unsubstituted heteroalkyl, substituted or unsubstituted aryl, substituted or unsubstituted heteroaryl, and substituted or unsubstituted heterocyclyl groups.

The term “pharmaceutically acceptable salts” includes salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain compounds of the present invention contain both basic and acidic functionalities that allow the compounds to be converted into either base or acid addition salts.

The neutral forms of the compounds are preferably regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

In addition to salt forms, the present invention contemplates compounds that are in a prodrug form. Prodrugs of the compounds described herein are those compounds that readily undergo chemical changes under physiological conditions to provide compounds having the inhibitory activity desired within the present invention. Thus, prodrugs can undergo more than one chemical change under physiological conditions to provide an inhibitory activity. Additionally, prodrugs can be converted to compounds having the desired inhibitory activity by chemical or biochemical methods in an ex vivo environment. For example, prodrugs can be slowly converted to compounds having the desired inhibitory activity within the present invention when placed in a transdermal patch reservoir with a suitable enzyme or chemical reagent.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

Certain compounds of the present invention possess asymmetric carbon atoms (optical centers) or double bonds; the racemates, diastereomers, geometric isomers and individual isomers are encompassed within the scope of the present invention.

The compounds of the invention are prepared as a single isomer (e.g., enantiomer, cis-trans, positional, diastereomer) or as a mixture of isomers. In a preferred embodiment, the compounds are prepared as substantially a single isomer. Methods of preparing substantially isomerically pure compounds are known in the art. For example, enantiomerically enriched mixtures and pure enantiomeric compounds can be prepared by using synthetic intermediates that are enantiomerically pure in combination with reactions that either leave the stereochemistry at a chiral center unchanged or result in its complete inversion. Alternatively, the final product or intermediates along the synthetic route can be resolved into a single stereoisomer. Techniques for inverting or leaving unchanged a particular stereocenter, and those for resolving mixtures of stereoisomers are well known in the art and it is well within the ability of one of skill in the art to choose an appropriate method for a particular situation. See, generally, Furniss et al. (eds.),VOGEL'S ENCYCLOPEDIA OF PRACTICAL ORGANIC CHEMISTRY 5^{TH} ED., Longman Scientific and Technical Ltd., Essex, 1991, pp. 809-816; and Heller, Acc. Chem. Res. 23: 128 (1990).

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present invention, whether radioactive or not, are intended to be encompassed within the scope of the present invention.

The term “cleaveable group” refers to a moiety that is unstable in vivo. Preferably, cleaving the “cleaveable group” allows for activation of the therapeutic agent. Operatively defined, the group is preferably cleaved in vivo by the biological environment. The cleavage may come from any process without limitation, e.g., enzymatic, reductive, pH, etc. Preferably, the cleaveable group is selected so that activation occurs at the desired site of action, which can be a site in or near the target cells (e.g., carcinoma cells) or tissues such as at the site of therapeutic action. When such cleavage is enzymatic, exemplary enzymatically cleaveable groups include natural amino acids or peptide sequences that end with a natural amino acid, and are attached at their carboxyl terminus to the linker. While the rate of cleavage is not critical to the invention, preferred examples of cleaveable groups are those in which at least about 10% of the cleaveable groups are cleaved in the body within 24 hours of administration, most preferably at least about 35%. Preferred cleaveable groups are peptide bonds, ester linkages, and disulfide linkages.

The terms “polypeptide,” and “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer. These terms also encompass the term “antibody.”

The terms “amino acid” “amino acid residue” refer to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but functions in a manner similar to a naturally occurring amino acid. The term “unnatural amino acid” is intended to represent the “D” stereochemical form of the twenty naturally occurring amino acids described above. It is further understood that the term unnatural amino acid includes homologues of the natural amino acids, and synthetically modified forms of the natural amino acids. The synthetically modified forms include, but are not limited to, amino acids having alkylene chains shortened or lengthened by up to two carbon atoms, amino acids comprising optionally substituted aryl groups, and amino acids comprised halogenated groups, preferably halogenated alkyl and aryl groups. When incorporated into a compound of the invention, the amino acid is in the form of an “amino acid side chain” or “amino acid residue.” In an exemplary embodiment, the carboxylic acid group of the amino acid has been replaced a —C(O)—, which is the locus of attachment for the amino acid residue to the remainder of the molecule. Thus, for example, an alanine side chain is —C(O)—CH(NH₂)—CH₃, and so forth.

Inhibitors of IMPDH

Compounds that inhibit IMPDH are described in literature and in patents. For example, mycophenolic acid (“MPA”) was initially described as a weakly-active antibiotic found in the fermentation broth of Penicillium brevicompactum. A related compound, mycophenolate mofetil (the morpholinoethyl ester of MPA), also inhibits IMPDH. Both MPA and mycophenolate mofetil have been used as immunosuppressant drugs. See, for example, U.S. Pat. Nos. 3,880,995; 4,727,069; 4,753,935; and 4,786,637, all incorporated herein by reference.

Other IMPDH inhibitors include Tiazofurin. Tiazofurin is anabolized to become an NAD analog that inhibits IMPDH. Tiazofurin may be prepared as described in U.S. Pat. No. 4,680,285 or U.S. Pat. No. 4,451,648, incorporated herein by reference.

Ribavirin, another IMPDH inhibitor is a nucleoside prodrug and inhibits by binding to the IMP site of the enzyme. Ribavirin may be prepared as described in U.S. Pat. No. 4,138,547 or U.S. Pat. No. 3,991,078, incorporated herein by reference. Ribavirin is currently in use as an antiviral agent. A prodrug of Ribavirin, Viramidine, is also available. Ribavirin has been proposed as an anticancer agent in combination with the IMPDH inhibitor, Tiazofurin. See, e.g., U.S. Pat. No. 5,405,837.

The compound mizoribine is also an effective inhibitor of IMPDH. Mizoribine was originally discovered in the culture broth of Eupenicillium brefeldianum M-2116. Mizoribine is a prodrug and is not incorporated into cellular nucleic acids. Mizoribine is phosphorylated by the enzyme adenosine kinase (AK) and then converted to its active form: mizoribine-5′monophosphate. The phosphorylated active form of mizoribine inhibits IMPDH by binding to the IMP site. Ishikawa, H. Current Med. Chem. 6:575-597 (1999). Various processes are known for producing mizoribine, e.g. J. Antibiotics, 27, (10) 775 (1974), Chem. Pharm. Bull., 23, 245 (1975), Japanese Patent Laid-open (ko-kai) Nos. 56894/1973, 1693/1976, 121275/1975, 121276/1975, and the like. Mizoribine is currently used as an immunosuppressant. See e.g. U.S. Pat. Nos. 5,472,947 and 5,462,929.

Mizoribine aglycone, also a prodrug, is used as an IMPDH inhibitor. Like mizoribine, the active form of mizoribine aglycone is mizoribine-5′ monophosphate, which inhibits IMPDH. Mizoribine aglycone is converted to its active form by the intracellular enzyme adenosine phosphoribosyltransferase (APRT). See e.g., Fukai et al., Cancer Research 42:1098-1102 (1982).

Inhibitors of IMPDH in Combination With Inhibitors of α-Tubulin Polymerization

According to the present invention, combinations of IMPDH inhibitors and inhibitors of α-tubulin polymerization are used to treat cancer. α-tubulin binds to and hydrolyzes GTP. GTP-bound α-tubulin dimers promote polymerization of the protein into microtubules, while GDP-bound α-tubulin dimers promote disassembly. Thus, α-tubulin turnover is sensitive to levels of GTP in the cell. Without wishing to be bound by theory, it is believed that cancer cells with high rates of α-tubulin polymerization and depolymerization are especially sensitive to IMPDH inhibitors because processes of cell division, growth, and/or movement, upon which a cancer cell relies for its continued existence, depend on α-tubulin polymerization, which, in turn, is dependent on GTP levels. This sensitivity is exacerbated through addition of compounds that inhibit microtubule polymerization.

In a preferred embodiment, a cancer treated with a combination of an IMPDH inhibitor and an inhibitor of α-tubulin polymerization is a slow growing cancer having a high rate of α-tubulin turnover. In proliferating cells (e.g., fast growing cells) α-tubulin polymerization is increased to promote microtubule growth required for spindle formation and entry into mitosis. However, since α-tubulin polymerization is required for other processes such as cell motility, high rates of α-tubulin polymerization can occur at times other than mitosis, even in cells that are proliferating slowly. At those times, however, microtubules are unstable because α-tubulin depolymerization is occurring at the same time, e.g., the α-tubulin turnover rate is high. Thus, slow growing cells can have a high rate of α-tubulin turnover, even though microtubule formation is not readily apparent. In slow growing malignant cells, high rates of α-tubulin turnover promote cellular processes such as chemokinesis and chemotaxis. In some instances treatment of slow growing malignant cells with an inhibitor of α-tubulin polymerization results in apoptosis. See e.g., Leoni, et al., J. Natl. Cancer Inst. 92:217-224 (2000); Hua, et al., Cancer Res. 61:7248-7254 (2001); and U.S. Pat. No. 6,162,810. Assays to measure α-tubulin turnover and, thus, cancer cells having a high rate of α-tubulin turnover, are known to those of skill in the art.

Examples of inhibitors of α-tubulin polymerization are known. In a preferred embodiment, inhibitors are chosen that interact with α-tubulin at or near the GTP binding site.

Indanone and tetralone compounds are known to inhibit α-tubulin polymerization. See e.g., U.S. Pat. No. 6,162,810. In a preferred embodiment, the indanone compound indanocine is used to inhibit microtubule polymerization.

Vinca alkaloids are known to inhibit oα-tubulin polymerization. Examples of vinca alkaloids include vincristine, vinblastine and vinorelbine. Methods to make and use vincristine and vinblastine are known. See, e.g., U.S. Pat. Nos. 3,097,137 and 3,205,220.

Vinorelbine, also known as Navelbine, is also used to inhibit α-tubulin polymerization. See e.g., Gregory, R. K. and Smith, I. E. Br. J. Cancer 82:1907-1913 (2000).

Combretastatin-A is known to inhibit α-tubulin polymerization. See, e.g., U.S. Pat. Nos. 4,996,237 and 5,561,122.

Colchicine is also known to inhibit α-tubulin polymerization. Colchicine is commonly used to treat gout. A description and methods to use colchicine are found for example in Insel, P. Analgesic-Antipyretic and Antiinflammatory Agents and Drugs Employed in the Treatment of Gout, in Goodman and Gilman's The Pharmacological Basis of Therapeutics 9, 647-649 (Hardman, J., et al. eds. 1996).

Measurement of Rates of α-Tubulin Turnover

In some embodiments, the combination of an IMPDH inhibitor and an α-tubulin polymerization inhibitor are used to treat a slow growing cancer. As used herein a “slow growing cancer” is a cancer that is present in a subject in need of treatment, wherein the subject has more than a 50% survival rate after 5 years, even if at the time of diagnosis, the cancer has spread to the regional lymph nodes. Slow growing cancers can include the following: chronic lymphocytic leukemia (CLL), chronic myelogenous leukemia, non-Hodgkins lymphoma, multiple myeloma, chronic granulocytic leukemia, cutaneous T cell lymphoma, low grade lymphomas, colon cancer, uterine cancer, breast cancer, prostate cancer, and thyroid cancer.

In a preferred embodiment, the slow growing cancer treated with the combination of an IMPDH inhibitor and an α-tubulin polymerization inhibitor has a high rate of α-tubulin turnover. Assays to measure α-tubulin turnover are known to those of skill in the art. In cells with a high α-tubulin turnover rate, addition of a α-tubulin depolymerization inhibitor (e.g. taxol or paclitaxol) promotes accumulation of α-tubulin in microtubules, which can then be separated from dimeric α-tubulin by centrifugation. Western blot analysis of soluble and particulate fractions is used to determine the relative amount of α-tubulin incorporated in microtubules. After addition of a α-tubulin depolymerization inhibitor, most of the α-tubulin (e.g., at least 60%, preferably 70%, more preferably 75, 80, 85, 90, 95, or 100%) from a cell with a high turnover rate will partition with the particulate fraction. In cells that do not have a high rate of turnover, addition of an α-tubulin depolymerization inhibitor will have a smaller effect on the partitioning of α-tubulin after centrifugation. That is, more α-tubulin will partition with the soluble fraction.

Inhibitors of IMPDH in Combination with Ara-G Compounds

The present invention provides a method of treating cancer with combinations of IMPDH inhibitors and precursors of 9-beta-D-arabinofuranosylguanine 5′-triphosphate (Ara-GTP). The lower level of GTP that results from inhibition of IMPDH makes the cell more susceptible to Ara-GTP uptake and, thus, potentiates the effects of Ara-GTP. Without wishing to be bound by theory, it is believed that the Ara-GTP taken up by the GTP deficient cell is incorporated into DNA leading to termination of replication. Ara-G, a precursor of Ara-GTP, is used to treat cancer. See e.g., U.S. Pat. Nos. 4,136,175; 5,492,897; 5,747,472; and 5,821,236. Nelarabine, a prodrug of Ara-G, is also used to treat cancer. See e.g., Kisor, et al., J. Clin Onc. 18:995-1003 (2000). Additional compounds that are useful precursors of Ara-GTP include other 6-substituted beta-D-arabinofuranosylpurines that are converted to guanosine analogs by either adenosine deaminase or xanthine oxidase. Other prodrugs of Ara-G include any 9-beta-D-arabinofuranosyl 2-amino, 6-substituted purine that is converted to ara-G by either adenosine deaminase or xanthine oxidase. Examples of 6-substitutions with these properties include hydrogen, halogens, methoxy, and amino.

In some instances precursors of Ara-GTP are converted to Ara-GTP through the activity of deoxycytidine kinase. Thus, in some embodiments, cancers with high levels of deoxycytidine kinase are selected for therapy combining IMPDH inhibitors and precursors of Ara-GTP.

Inhibitors of IMPDH in Combination With Deficiency of De Novopurine Biosynthesis

IMPDH inhibitors can be used to treat cancers with deficiencies in synthesis of adenine. Some cancer cells are deficient in the enzyme methylthioadenosine phosphorylase (MTAP), which converts methylthioadenosine (MTA), a product of the polyamine biosynthetic pathway, to adenine and 5-methylthioribose-1-phosphate. See e.g., U.S. Pat. Nos. 5,571,510; 5,942,393; and 6,210,917.

In cells lacking MTAP, compounds containing adenine/adenosine cannot be recycled because MTA is not converted to adenine and 5-methylthioribose-1-phosphate. Accordingly, the enzymes of the adenine salvage pathway are upregulated, including adenosine kinase (AK) and adenine phosphoribosyl transferase (APRT). As discussed previously, AK converts the prodrug mizoribine to an active IMPDH inhibitor and APRT converts mizoribine aglycone to an active IMPDH inhibitor. Thus, in cells lacking MTAP, the conversion of the prodrugs mizoribine and mizoribine aglycone to active IMPDH inhibitors is increased, which potentiates the effects of such drugs in MTAP deficient cells as compared to the effects of the drugs in MTAP competent cells.

Another method to increase the conversion of the prodrugs mizoribine and mizoribine aglycone to active IMPDH inhibitors is to inhibit a de novo cellular pathway of adenine biosynthesis. Thus, combination of an IMPDH inhibitor and an inhibitor of de novo adenine biosynthesis can be used to treat cancer cells. In a preferred embodiment, the enzyme adenylsuccinate synthetase (ASS) is inhibited by the compound L-alanosine. See e.g. U.S. Pat. Nos. 5,840,505; 6,210,917; and 6,214,571. The combination of inhibitors of IMPDH and inhibitors of de novo purine biosynthesis can be used to treat cancer cells that are deficient in MTAP activity and can also be used to treat cancer cells that are not deficient in MTAP activity.

Other compounds that inhibit the de novo pathway of purine biosynthesis are also encompassed by the present invention. For example, folate is incorporated into purine molecules during de novo biosynthesis. Thus, inhibitors of folate metabolism (e.g., antifolates) can inhibit de novo purine biosynthesis. Antifolate inhibitors of de novo purine biosynthesis include methotrexate and trimetrexate, which inhibit the enzyme dihydrofolate reductase, an important enzyme in folate metabolism. Methotrexate is currently used for cancer chemotherapy and trimetrexate is currently used for antiparasitic therapy. See e.g., Chabner, B. A. et al., Antineoplastic Agents, in Goodman and Gilman's The Pharmacological Basis of Therapeutics 9, 1243-1247 (Hardman, J., et al. eds. 1996). Antifolate inhibitors affect enzymes that synthesize precursors of purine biosynthesis, including folylpolyglutamate synthetase(FPGS). FPGS inhibitors include 10-propargyl-5,8-dideazafolic acid (PDDF) and N-[5-[N-(3,4-dihydro-2-methyl-4-oxoquinazolin-6-ylmethyl)-N-methylamino]-2-thenoyl]-L-glutamic acid (ZD1694, Tomudex). Multi-targeted antifolates are also known, e.g., N-[4-[2-(2-amino-3,4-dihydro-4-oxo-7H-pyrrolo[2,3-d]-pyrimidin-5-yl)ethyl]-benzoyl]-L-glutamic acid (LY231514).

Two enzymes of the purine biosynthesis pathway incorporate folate into purines and can be inhibited by specific antifolates. The enzyme glycinamide ribonucleotide formyltransferase (GARFT) is inhibited by the antifolates 6-(2′-formyl-2′naphthyl-ethyl)-2-amino-4(3H)-oxoquinazoline (LL95509), (6R,S)-5,10-dideazatetrahydrofolic acid (DDATHF), and 4-[2-(2-amino-4-oxo-4,6,7,8-tetrahydro-3Hpyrimidino[5,4,6][1,4]-thiazin-6yl)-(S)-ethyl]-2,5-thienoylamino-L-glutamic acid (AG2034). The enzyme aminoimidazolecarboxamide ribonucleotide formyltransferase (AICARFT) is inhibited by the antifolate N-[5-(2-[(2,6-diamino-4(3H)-oxopyrimidin-5-yl)thio]ethyl)thieno-2-yl]-L-glutamic acid (AG2009).

Inhibitors of IMPDH can also be used in combination with inhibitors of the salvage pathway of ATP biosynthesis to treat cancer. In one embodiment, inhibitors of the enzyme adenosine kinase are used in combination with inhibitors of IMPDH. Inhibitors of adenosine kinase include N7-((1′R,2′S,3′R,4′S)-2′,3′-dihydroxy-4′-amino-cyclopentyl)-4-amino-5-bromo-pyrrolo[2,3-a]pyrimidine, 5′-aminotubercidin, 5-amino-5′-deoxyadenosine, 5′-deoxy-5′-amino-clitocine, 4-amino-5-(3-bromophenyl)-7-(6-morpholino-pyridin-3-yl)pyrido[2,3-d]pyrimidine, 5-iodotubercidin (5-IT), and 5′-deoxy,5-iodotubercidin (5′d-5IT).

Detection of Cells That are Deficient in MTAP Activity

Those of skill in the art will recognize that MTAP deficient cancer cells can be identified using standard molecular and biochemical techniques. For example, a sample of cancer cells can be obtained and assayed for catalytic activity of the MTAP enzyme. See, e.g. Seidenfeld et al., Biochem. Biophys. Res. Commun. 95: 1861-1866 (1980). The MTAP catalytic activity is compared to that of an untransformed cell sample to determine whether MTAP activity is deficient.

MTAP deficiency can also be determined by immunoassays to measure protein levels or by using nucleic acid probes or PCR technology to determine DNA or mRNA levels. See, e.g., U.S. Pat. Nos. 5,571,510; 5,942,393; and 6,210,917; herein incorporated by reference. Levels of MTAP protein and MTAP nucleic acid are compared to levels in untransformed control cells to determine if the cancer cells are deficient in MTAP.

The nucleic acid sequence of the MTAP gene is known. Briefly, MTAP nucleic acid levels can be measured using hybridization technology. Southern hybridization can be used to detect rearrangements or deletions of the gene locus encoding MTAP. Northern hybridization can be used to determine levels of MTAP mRNA present in cancer cell.

Those skilled in the art will also recognize that other detection means to detect the presence or absence of MTAP nucleic acids in cells. For example, using the nucleic acid sequence of MTAP, one of skill in the art could construct oligonucleotide probes which would hybridize to MTAP DNA present in a cell sample. Conversely, because it is believed that MTAP deficiency results from the genomic deletion of the gene which would encode the MTAP protein, it can be assumed that if no gene encoding MTAP is detected in a cell sample that the cells are MTAP negative.

Levels of MTAP protein can be determined using immunological assays. Antibodies which are specific for MTAP are produced by immunization of a non-human with antigenic MTAP or MTAP peptides. Generally, the antigenic MTAP peptides may be isolated and purified from mammalian tissue according to the method described by Ragnione, et al., J. Biol. Chem., 265: 6241-6246 (1990), or can be made by recombinant or synthetic means.

Once antigenic MTAP or MTAP peptides are obtained, antibodies to the immunizing peptide are produced by introducing peptide into a mammal (such as a rabbit, mouse or rat). A multiple injection immunization protocol is preferred for use in immunizing animals with the antigenic MTAP peptides (see, e.g., Langone, et al., eds., “Production of Antisera with Small Doses of Immunogen: Multiple Intradermal Injections”, Methods of Enzymology (Acad. Press, 1981)). For example, a good antibody response can be obtained in rabbits by intradermal injection of 1 mg of the antigenic MTAP peptide emulsified in Complete Freund's Adjuvant followed several weeks later by one or more boosts of the same antigen in Incomplete Freund's Adjuvant.

If desired, the immunizing peptide may be coupled to a carrier protein by conjugation using techniques which are well-known in the art. Such commonly used carriers which are chemically coupled to the peptide include keyhole limpet hemocyanin (KLH), thyroglobulin, bovine serum albumin (BSA), and tetanus toxoid. The coupled peptide is then used to immunize the animal (e.g. a mouse or a rabbit). Because MTAP is presently believed to be conserved among mammalian species, use of a carrier protein to enhance the immunogenicity of MTAP proteins is preferred.

Polyclonal antibodies produced by the immunized animals can be further purified, for example, by binding to and elution from a matrix to which the peptide to which the antibodies were raised is bound. Those of skill in the art will know of various techniques common in the immunology arts for purification and/or concentration of polyclonal antibodies, as well as monoclonal antibodies (see, for example, Coligan, et al., Unit 9, Current Protocols in Immunology, Wiley Interscience, 1991).

For their specificity and ease of production, monoclonal antibodies are preferred for use in detecting MTAP negative cells. For preparation of monoclonal antibodies, immunization of a mouse or rat is preferred. The term “antibody” as used in this invention is meant also to include intact molecules as well as fragments thereof, such as for example, Fab and F(ab′).sub.2′ which are capable of binding the epitopic determinant. Also, in this context, the term “mAb's of the invention” refers to monoclonal antibodies with specificity for MTAP.

The general method used for production of hybridomas secreting monoclonal antibodies (“mAb's”) is well known (Kohler and Milstein, Nature, 256:495, 1975). Briefly, as described by Kohler and Milstein, the technique comprised isolation of lymphocytes from regional draining lymph nodes of five separate cancer patients with either melanoma, teratocarcinoma or cancer of the cervix, glioma or lung. The lymphocytes were obtained from surgical specimens, pooled, and then fused with SHFP-1. Hybridomas were screened for production of antibody which bound to cancer cell lines. An equivalent technique can be used to produce and identify mAb's with specificity for MTAP.

Confirmation of MTAP specificity among mAbs of the invention can be accomplished using relatively routine screening techniques (such as the enzyme-linked immunosorbent assay, or “ELISA”) to determine the elementary reaction pattern of the mAb of interest.

It is also possible to evaluate an mAb to determine whether it has the same specificity as a mAb of the invention without undue experimentation by determining whether the mAb being tested prevents a mAb of the invention from binding to MTAP. If the mAb being tested competes with the mAb of the invention, as shown by a decrease in binding by the mAb of the invention, then it is likely that the two monoclonal antibodies bind to the same or a closely related epitope.

Still another way to determine whether a mAb has the specificity of a mAb of the invention is to pre-incubate the mAb of the invention with an antigen with which it is normally reactive, and determine if the mAb being tested is inhibited in its ability to bind the antigen. If the mAb being tested is inhibited then, in all likelihood, it has the same, or a closely related, epitopic specificity as the mAb of the invention.

Once suitable antibodies are obtained as described above, they are used to detect MTAP in a malignancy. However, those skilled in the immunological arts will recognize that MTAP may be detected using the antibodies described above in immuno-blot assays or other immunoassay formats, in either liquid or solid phase (when bound to a carrier).

Detection of MTAP using anti-MTAP antibodies can be done utilizing immunoassays which are run in either the forward, reverse, or simultaneous modes, including immunohistochemical assays on physiological samples. Suitable immunoassay protocols include competitive and non-competitive protocols performed in either a direct or indirect format. Examples of such immunoassays are the radioimmunoassay (RIA) and the sandwich (immunometric) assay. Those of skill in the art will know, or can readily discern, other immunoassay formats without undue experimentation.

In addition, the antibodies utilized in the immunoassays may be detectably labeled. A label is a substance which can be covalently attached to or firmly associated with a nucleic acid probe which will result in the ability to detect the probe. For example, a level may be radioisotope, an enzyme substrate or inhibitor, an enzyme, a radiopaque substance (including colloidal metals), a fluorophore, a chemiluminescent molecule, liposomes containing any of the above labels, or a specific binding pair member. A suitable label will not lose the quality responsible for detectability during amplification.

Those skilled in the diagnostic art will be familiar with suitable detectable labels for use in in vitro detection assays. For example, suitable radioisotopes include ³H, ¹²⁵I, ¹³¹I, ³²P, ¹⁴C, and ³⁵S. Radiolabeled antibodies can be detected directly by gamma counter or by densitometry of autoradiographs. Examples of suitable chemiluminescent molecules are acridines or luminol. Examples of suitable fluorophores are fluorescein, phycobiliprotein, rare earth chelates, dansyl or rhodamine.

Examples of suitable enzyme substrates or inhibitors are compounds which will specifically bind to horseradish peroxidase, glucose oxidase, glucose-6-phosphate dehydrogenase, β-galactosidase, pyruvate kinase or alkaline phosphatase acetylcholinesterase. Examples of radiopaque substance are colloidal gold or magnetic particles.

A specific binding pair comprises two different molecules, wherein one of the molecules has an area on its surface or in a cavity which specifically binds to a particular spatial and polar organization of another molecule. The members of the specific binding pair are often referred to as a ligand and receptor or ligand and anti-ligand. For example, if the receptor is an antibody the ligand is the corresponding antigen. Other specific binding pairs include hormone-receptor pairs, enzyme substrate pairs, biotin-avidin pairs and glycoprotein-receptor pairs. Included are fragments and portions of specific binding pairs which retain binding specificity, such as fragments of immunoglobulins, including Fab fragments and the like. The antibodies can be either monoclonal or polyclonal. If a member of a specific binding pair is used as a label, the preferred separation procedure will involve affinity chromatography.

The antibodies may also be bound to a carrier. Examples of well-known carriers include glass, polystyrene, polypropylene, polyethylene, dextran, nylon, amyloses, natural and modified celluloses, polyacrylamides, agaroses and magnetite. The nature of the carrier can be either soluble or insoluble for purposes of the invention. Those skilled in the art will know of other suitable carriers for binding antibodies, or will be able to ascertain such, using routine experimentation.

Inhibitors of IMPDH in Combination With Inhibitors of Receptor Tyrosine Kinases

Inhibitors of IMPDH can be combined with inhibitors of receptor tyrosine kinases to treat cancer. Cellular GTP levels are regulated in tandem with cellular ATP levels because the enzyme nucleoside diphosphate synthase functions to equilibrate the levels of both. Thus, when GTP levels fall, ATP levels will also fall. Because administration of IMPDH inhibitors lowers cellular GTP levels, cellular ATP levels will also decrease. Administration of an IMPDH inhibitor will allow therapeutically beneficial manipulation of ATP levels, in addition to therapeutically beneficial manipulation of GTP levels.

Recently, inhibitors of receptor tyrosine kinases have been approved or entered clinical trials for treatment of cancer. Receptor tyrosine kinases catalyze the phosphorylation of tyrosine residues on proteins using ATP as a substrate. Many of the kinase inhibitors that are approved or under development are competitive inhibitors with respect to ATP. Without wishing to be bound by theory, it is believed that co-administration of IMPDH inhibitors will lower both cellular GTP and ATP concentrations. The lowered ATP concentrations will make less ATP available as substrate for the receptor tyrosine kinase and, thus, increase the inhibitory effects of the tyrosine kinase inhibitor molecules and enhance their therapeutic potential.

Gleevec, also known as imatinib mesylate or ST1571, is a kinase inhibitor that can be used in combination with IMPDH inhibitors to treat cancer. Gleevec is a low molecular weight molecule known to inhibit the following tyrosine kinases: Bcr-Abl, Abl, PDGFR, and c-kit. The kinases are believed to act in intracellular signaling pathways that affect cell proliferation, adhesion and survival. Gleevec blocks the binding of ATP to a kinase molecule, and in some instances prevents transduction of signals that stimulate cell proliferation or cell survival, leading to malignancy. See e.g., Shawver, L. K., et al., Cancer Cell 1:117-123 (2002); Drucker, B. J. Trends, Mol. Med. 8:S-14-S-18 (2002). Gleevec has been successfully used to treat the following cancers: chronic myelogenous leukemia and gastrointestinal stromal tumor. Id. Methods to make and use Gleevec are know to those of skill in the art. See e.g., U.S. Pat. No. 6,306,874.

Another class of kinase inhibitors inhibits receptor tyrosine kinases of the Epidermal Growth Factor Receptor (EGFR) family, e.g. ErbB1, ErbB2, ErbB3, and ErbB4. EGFR proteins are expressed in a wide variety of tissues. See e.g., de Bono J. S. and Rowinsky, E. K. Trends Mol. Med. 8:S19-S26 (2002). Signaling through EGFR family members activates transduction pathways that stimulate cellular proliferation, migration, neovascularization, and resistance to cell death enhancing signals. EGFR protein overexpression occurs in many cancers including the following: head and neck, non-small cell lung cancer (NSCLC), laryengeal, esophageal, gastric, pancreatic, colon, renal cell, bladder, breast, ovarian, cervical, prostate, papillary thyroid cancers, melanoma, and gliomas. See e.g., Shawver, L. K., et al., Cancer Cell 1: 117-123 (2002).

Iressa, a quinazoline also known as ZD1839, is a small molecule that inhibits the tyrosine kinase activity of ErbB1 by competing with ATP for binding to the enzyme. Iressa has been used to treat NSCLC. Other small molecule inhibitors of the ErbB1 kinase are known including: OSI-774 and PKI 116. Some small molecule inhibitors inhibit more than one EGFR protein e.g., GW2016, EKB-569, and CI1033 (PD183805). Methods to make and use inhibitors of EGFR protein tyrosine kinases are known to those of skill in the art. See e.g., Shawver, L. K., et al., Cancer Cell 1:117-123 (2002); de Bono J. S. and Rowinsky, E. K. Trends Mol. Med. 8:S19-S26 (2002).

Gleevec, Iressa, and the other listed receptor tyrosine kinase inhibitors also inhibit in vitro the growth of many different cancers, in addition to those listed above. However, the in vivo use of receptor tyrosine kinase inhibitors for treatment of many cancers is restricted because therapeutically effective doses also have deleterious effects on normal cells, especially bone marrow cells and GI epithelium. IMPDH inhibitors deplete cells of ATP and GTP, thereby lessening the substrates available for receptor tyrosine kinases. Decreased levels of substrates will allow inhibitors of receptor tyrosine kinases to be used at lower therapeutically effective dosages, when combined with IMPDH inhibitors.

A list of cancers that can be treated with the combination of IMPDH inhibitors and inhibitors of receptor tyrosine kinases follows: adult and pediatric acute myeloid leukemias (AML); chronic myeloid leukemia (CML; acute lymphocytic leukemia (ALL; chronic lymphocytic leukemia (CLL; hairy cell leukemia; secondary leukemia; acute nonlymphocytic leukemia; chronic lymphocytic leukemia; acute granulocytic leukemia; chronic granulocytic leukemia; acute promyelocytic leukemia; adult T-cell leukemia; aleukemic leukemia; a leukocythemic leukemia; basophylic leukemia; blast cell leukemia; bovine leukemia; chronic myelocytic leukemia; leukemia cutis; embryonal leukemia; eosinophilic leukemia; Gross' leukemia; hairy-cell leukemia; hemoblastic leukemia; hemocytoblastic leukemia; histiocytic leukemia; stem cell leukemia; acute monocytic leukemia; leukopenic leukemia; lymphatic leukemia; lymphoblastic leukemia; lymphocytic leukemia; lymphogenous leukemia; lymphoid leukemia; lymphosarcoma cell leukemia; mast cell leukemia; megakaryocytic leukemia; micromyeloblastic leukemia, monocytic leukemia; myeloblastic leukemia; myelocytic leukemia; myeloid granulocytic leukemia; myelomonocytic leukemia; Naegeli leukemia; plasma cell leukemia; plasmacytic leukemia; promyelocytic leukemia; Rieder cell leukemia; Schilling's leukemia; stem cell leukemia; subleukemic leukemia; undifferentiated cell leukemia; non-Hodgkin's lymphoma (NHL); Hodgkin's disease; myelodysplastic syndromes (MDS); myeloproliferative syndromes (MPS); myelomas, such as solitary myeloma and multiple myeloma; skin cancers, including melanomas, basal cell carcinomas, Kaposi's sarcoma, and squamous cell carcinomas; epithelial carcinomas of the head and neck; lung cancers, including squamous or epidermoid carcinoma, small cell carcinoma, adenocarcinoma, and large cell carcinoma; breast cancer, including invasive breast cancer and non-invasive breast cancer; gastrointestinal tract cancers, including esophageal cancers, gastric adenocarcinoma, primary gastric lymphoma, colorectal cancer, small bowel tumors and cancers of the anus; pancreatic cancer and cancers of the liver, including hepatocellular cancer; bladder cancer; renal cell carcinoma; prostatic carcinoma; testicular cancer; ovarian cancer, carcinoma of the fallopian tube; uterine cancer; cervical cancer; sarcomas of the bone and soft tissue, including osteosarcoma, chondrosarcoma, and Ewing's sarcoma; and malignant tumors of the thyroid, including papillary, follicular, and anaplastic carcinomas.

Inhibitors of IMPDH in Combination with G-Protein Coupled Receptor Antagonists

Another major class of cancer treatment targets that are regulated by cellular GTP levels are the G protein coupled receptors (GPCRs). Approximately 2000 genes encoding GPCRs are found in the genome, and several GPCR genes are selectively expressed by cancers.

GPCRs share a common structure: seven membrane spanning domains, an extracellular domain and an intracellular domain. After ligand binding, the conformation of the GPCR changes and the intracellular domain activates a specific G-protein, either directly or through activation of a guanine nucleotide exchange factor (GEF), which activates another G-protein. See e.g., Dhanasekaran, N., et al., Endocrine Reviews 16:259-270 (1995) and Healy, D. P., Meth. Enz. 343:448-459 (2002).

G proteins are heterotrimeric proteins that switch from an inactive, GDP-bound state to an active GTP-bound state. See e.g., Dhanasekaran, N., et al., Endocrine Reviews 16:259-270 (1995). GTP-bound G-proteins activate signal transduction pathways. Without wishing to be bound by theory, it is believed that activation of G-proteins is dependant on the cellular ratio of GTP to GDP. The ratio of GTP to GDP governs the activity of these GPCRs: GTP in the binding site is “on” signal and GDP is an “off” signal. The IMPDH inhibitors may be able to lower the GTP/GDP ratio to increase the off/on ratio and thus potentiate the effects of GPCR antagonists. IMPDH inhibitors, which affect GTP and GDP levels will diminish the ability of G-proteins to be activated. G-proteins affect the growth of cancer cells by activating signal transduction pathways that lead to cellular proliferation or increased survival of cancer cells, e.g., increasing ability to metastasize, to promote blood vessel growth and nutrient uptake, or decreasing susceptibility to apoptosis, for example.

In some cancers the expression of GPCRs is increased, leading to increased activation of G-proteins and their associated signal transduction pathways. Thus, the combination of IMPDH inhibitors and antagonists of GPCRs can be used to treat those cancers more effectively than treatment with either agent alone. In addition, some G-proteins downstream of GCPRs are oncogenes, and cancers with increased activity of downstream G-proteins can also be effectively treated by the combination of IMPDH inhibitors and antagonists of GPCR's. One example of an oncogene downstream of a GPCR is the Rho oncogene. See e.g., Seasholtz, T. M., et al., Mole. Pharm. 55:949-956 (1999).

Because GPCR's are widely expressed, the combination of an IMPDH inhibitor and a GPCR can be used to treat a variety of cancers. Such cancers include adult and pediatric acute myeloid leukemias (AML); chronic myeloid leukemia (CML; acute lymphocytic leukemia (ALL; chronic lymphocytic leukemia (CLL; hairy cell leukemi; secondary leukemia; acute nonlymphocytic leukemia; chronic lymphocytic leukemia; acute granulocytic leukemia; chronic granulocytic leukemia; acute promyelocytic leukemia; adult T-cell leukemia; aleukemic leukemia; a leukocythemic leukemia; basophylic leukemia; blast cell leukemia; bovine leukemia; chronic myelocytic leukemia; leukemia cutis; embryonal leukemia; eosinophilic leukemia; Gross' leukemia; hairy-cell leukemia; hemoblastic leukemia; hemocytoblastic leukemia; histiocytic leukemia; stem cell leukemia; acute monocytic leukemia; leukopenic leukemia; lymphatic leukemia; lymphoblastic leukemia; lymphocytic leukemia; lymphogenous leukemia; lymphoid leukemia; lymphosarcoma cell leukemia; mast cell leukemia; megakaryocytic leukemia; micromyeloblastic leukemia, monocytic leukemia; myeloblastic leukemia; myelocytic leukemia; myeloid granulocytic leukemia; myelomonocytic leukemia; Naegeli leukemia; plasma cell leukemia; plasmacytic leukemia; promyelocytic leukemia; Rieder cell leukemia; Schilling's leukemia; stem cell leukemia; subleukemic leukemia; undifferentiated cell leukemia; non-Hodgkin's lymphoma (NHL); Hodgkin's disease; myelodysplastic syndromes (MDS); myeloproliferative syndromes (MPS); myelomas, such as solitary myeloma and multiple myeloma; skin cancers, including melanomas, basal cell carcinomas, Kaposi's sarcoma, and squamous cell carcinomas; epithelial carcinomas of the head and neck; lung cancers, including squamous or epidermoid carcinoma, small cell carcinoma, adenocarcinoma, and large cell carcinoma; breast cancer, including invasive breast cancer and non-invasive breast cancer; gastrointestinal tract cancers, including esophageal cancers, gastric adenocarcinoma, primary gastric lymphoma, colorectal cancer, small bowel tumors and cancers of the anus; pancreatic cancer and cancers of the liver, including hepatocellular cancer; bladder cancer; renal cell carcinoma; prostatic carcinoma; testicular cancer; ovarian cancer, carcinoma of the fallopian tube; uterine cancer; cervical cancer; sarcomas of the bone and soft tissue, including osteosarcoma, chondrosarcoma, and Ewing's sarcoma; and malignant tumors of the thyroid, including papillary, follicular, and anaplastic carcinomas. GPCR's are upregulated in some prostate cancers and have been targeted therapeutically. For example, the somatostatin and gonadotropin releasing hormone receptors targeted in prostate cancer are GPCRs. Antagonists of the gonadotropin releasing hormone receptor include leuprolide and goserelin. Antagonists of the somatostatin receptor include octreotide. See e.g., Erlichman, C. and Loprinzi, C., Hormonal Therapies, in Cancer: Principles and Practice of Oncology, 5:395-405 (DeVita, V. et al., eds. 1997). The endothelin-A receptor is upregulated in metastatic bone cancer associated with prostate cancer and is treated with the GPCR antagonist atrasentan. See e.g., Carducci, M. A., et al., J. Clin. Oncol. 20:2171-2180 (2002). Thus, IMPDH inhibitors can be used to treat cancers in combination with the GPCR antagonists atrasentan, leuprolide, goserelin, and octreotide.

Treatment of Cancer by Prolonged Administration of Mizoribine, Mizoribine Aglycone, or Prodrugs of those Compounds

One reason mizoribine and related IMPDH inhibitors may have failed in cancer treatment in the past is that they do not kill cells directly, but rather starve cells of GTP. Death by starvation is slower than direct killing of cells. Without wishing to be bound by theory, use of mizoribine and other IMPDH inhibitors can be optimized by administering the compounds in a manner designed to achieve high plasma levels over long periods of time.

Mizoribine has a short plasma half life of a few hours and is only given at 150 mg bid. For improved toxicity to cancer cells, IMPDH inhibitors including mizoribine or its aglycone can be administered in a manner designed to achieve higher plasma levels and/or for longer periods of time. Methods to achieve higher plasma levels of IMPDH inhibitors for longer periods of time include frequent administration schedules and administration of prodrugs that remain in the body for longer periods of time. For example, in one embodiment mizoribine or its aglycone can be administered on a schedule to produce a desired plasma concentration for a desired period of time. In another embodiment, mizoribine or its aglycone are administered as prodrugs that are effective for a prolonged period of time. Prodrugs of mizoribine or its aglycone include to compounds of Formula I.

In general, an appropriate dosage and treatment regimen provides the active compound(s) in an amount sufficient to provide therapeutic and/or prophylactic benefit, e.g., a therapeutically effective amount. Those of skill in the art will be able to determine a therapeutically effective amount of an IMPDH inhibitor. Therapeutically effective amounts of a compound can be determined using animal models and then extrapolated to human patients. A therapeutic response can also be monitored by establishing an improved clinical outcome (e.g., more frequent complete or partial remissions, or longer disease-free survival) in treated patients as compared to non-treated patients.

In one embodiment mizoribine, mizoribine aglycone, or prodrugs of those compounds are administered to a patient to yield a plasma level between 0.5 and 50 micromolar for between 6 and 72 hours. In another embodiment the plasma level of mizoribine, mizoribine aglycone, or prodrugs of those compounds of between 1 and 30 micromolar is maintained for between 8 and 48 hours. In a preferred embodiment, the plasma level of mizoribine, mizoribine aglycone, or prodrugs of those compounds is between 5 and 25 micromolar for between 10 and 24 hours. In a most preferred embodiment, the plasma level of mizoribine, mizoribine aglycone, or prodrugs of those compounds is greater than 10 micromolar for at least 12 hours.

Compositions and Formulations

For use in this invention, the active compound, e.g., IMPDH inhibitor, an α-tubulin polymerization inhibitor, an inhibitor of purine biosynthesis, or an inhibitor of a receptor tyrosine kinase is included or formulated into a composition for packing, storage, shipment and administration. In addition, racemic mixtures, enantiomers, prodrugs of either the racemic mixture or of a stereoisomer, a metabolite of either the racemic mixture or of a stereoisomer, or a salt of any of these, may be included in a formulation or composition. The compositions contain one or more pharmaceutically acceptable carrier and may also contain other therapeutically active ingredients as well as adjuvants and other ingredients that may be found in pharmaceutical compositions.

Thus, compounds of this invention can be formulated with a pharmaceutically acceptable carrier for administration to a subject. While any suitable carrier known to those of ordinary skill in the art may be employed in the pharmaceutical compositions of this invention, the type of carrier will vary depending on the mode of administration. The pharmaceutical composition is typically formulated such that the compound in question is present in a therapeutically effective amount, i.e., the amount of compound required to achieve the desired effect in terms of treating a subject.

For preparing pharmaceutical compositions, the pharmaceutically acceptable carriers can be either solid or liquid. Solid form preparations include powders, tablets, pills, capsules, cachets, suppositories, and dispersible granules. A solid carrier can be one or more substance that may also act as diluents, flavoring agents, binders, preservatives, tablet disintegrating agents, or an encapsulating material.

In powders, the carrier is a finely divided solid that is in a mixture with the finely divided active component. In tablets, the active component is mixed with the carrier having the necessary binding properties in suitable proportions and compacted in the shape and size desired.

Suitable carriers for the solid compositions of this invention include, for instance, magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. Alternatively the compositions may be prepared in a form with an encapsulating material as a carrier providing a capsule in which the active component, with or without other carriers, is surrounded by a carrier, which is thus in association with it. Similarly, cachets and lozenges are included. Tablets, powders, capsules, pills, cachets, and lozenges can be used as solid dosage forms suitable for oral administration.

Liquid form preparations include solutions, suspensions, and emulsions, for example, water or water/propylene glycol solutions or suspensions. Aqueous suspensions suitable for oral use can be made by dispersing the finely divided compound in water with viscous material, such as natural or synthetic gums, resins, methylcellulose, sodium carboxymethylcellulose, and other well-known suspending agents. For parenteral injection, liquid preparations can be formulated in solution in aqueous polyethylene glycol solution. In certain embodiments, the pharmaceutical compositions are formulated in a stable emulsion formulation (e.g., a water-in-oil emulsion or an oil-in-water emulsion) or an aqueous formulation that preferably comprises one or more surfactants. Suitable surfactants well known to those skilled in the art may be used in such emulsions. In one embodiment, the composition comprising the compound in question is in the form of a micellar dispersion comprising at least one suitable surfactant. The surfactants useful in such micellar dispersions include phospholipids. Examples of phospholipids include: diacyl phosphatidyl glycerols, such as: dimyristoyl phosphatidyl glycerol (DPMG), dipalmitoyl phosphatidyl glycerol (DPPG), and distearoyl phosphatidyl glycerol (DSPG); diacyl phosphatidyl cholines, such as: dimyristoyl phosphatidylcholine (DPMC), dipalmitoyl phosphatidylcholine (DPPC), and distearoyl phosphatidylcholine (DSPC); diacyl phosphatidic acids, such as: dimyristoyl phosphatidic acid (DPMA), dipalmitoyl phosphatidic acid (DPPA), and distearoyl phosphatidic acid (DSPA); and diacyl phosphatidyl ethanolamines such as: dimyristoyl phosphatidyl ethanolamine (DPME), dipalmitoyl phosphatidyl ethanolamine (DPPE), and distearoyl phosphatidyl ethanolamine (DSPE). Other examples include, but are not limited to, derivatives of ethanolamine (such as phosphatidyl ethanolamine, as mentioned above, or cephalin), serine (such as phosphatidyl serine) and 3′-O-lysyl glycerol (such as 3′-O-lysyl-phosphatidylglycerol).

Also included in compositions for use in this invention are solid form preparations that are intended to be converted, shortly before use, to liquid form preparations for oral administration. Such liquid forms include solutions, suspensions, and emulsions. These preparations may contain, in addition to the active compound, colorants, flavors, stabilizers, buffers, artificial and natural sweeteners, dispersants, thickeners, solubilizing agents, and the like.

The compositions of the invention may also be in the form of controlled release or sustained release compositions as known in the art, for instance, in matrices of biodegradable or non-biodegradable injectable polymeric microspheres or microcapsules, in liposomes, in emulsions, and the like.

Administration

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The compounds (in the form of their compositions) are administered to patients by the usual means known in the art, for example, orally or by injection, infusion, infiltration, irrigation, and the like. For administration by injection and/or infiltration or infusion, the compositions or formulations according to the invention may be suspended or dissolved as known in the art in a vehicle suitable for injection and/or infiltration or infusion. Such vehicles include isotonic saline, buffered or unbuffered and the like. Depending on the intended use, they also may contain other ingredients, including other active ingredients, such as isotonicity agents, sodium chloride, pH modifiers, colorants, preservatives, antibodies, enzymes, antibiotics, antifungals, antivirals, other anti-infective agents, and/or diagnostic aids such as radio-opaque dyes, radiolabeled agents, and the like, as known in the art. However, the compositions of this invention may comprise a simple solution or suspension of a compound or a pharmaceutically acceptable salt of a compound, in distilled water or saline.

Alternatively, the therapeutic compounds may be delivered by other means such as intranasally, by inhalation, or in the form of liposomes, nanocapsules, vesicles, and the like. Compositions for intranasal administration usually take the form of drops, sprays containing liquid forms (solutions, suspensions, emulsions, liposomes, etc.) of the active compounds. Administration by inhalation generally involves formation of vapors, mists, dry powders or aerosols, and again may include solutions, suspensions, emulsions and the like containing the active therapeutic agents

Routes and frequency of administration of the therapeutic compositions described herein, as well as dosage, will vary from individual to individual, and may be readily established using standard techniques. Preferably, between 1 and 100 doses may be administered over a 52-week period. A suitable dose is an amount of a compound that, when administered as described above, is capable of killing or slowing the growth of, cancers or cancer cells.

In general, an appropriate dosage and treatment regimen provides the active compound(s) in an amount sufficient to provide therapeutic and/or prophylactic benefit. Such a response can be monitored by establishing an improved clinical outcome (e.g., more frequent remissions, complete or partial, or longer disease-free survival) in treated patients as compared to non-treated patients.

A therapeutic amount of a compound described in this application, means an amount effective to yield the desired therapeutic response, for example, an amount effective to delay the growth of a cancer or to cause a cancer to shrink or not metastasize. If what is administered is not the compound (or compounds), but an enantiomer, prodrug, salt or metabolite of the compound (or compounds), then the term “therapeutically effective amount” means an amount of such material that produces in the patient the same blood concentration of the compound in question that is produced by the administration of a therapeutically effective amount of the compound itself. For instance, as shown in the examples below, a combination of 1 μM indanocine and 1 μM mizoribine has been shown to be effective against CLL cells. Accordingly, one therapeutically effective amount of indanocine and mizoribine is that which produces a blood concentration of 1 μM indanocine and at least 1 μM mizoribine in a patient. Similarly, if an enantiomer, prodrug or metabolite of the compositions, or a salt of the compositions or of any of these other compounds, is being administered, then one therapeutically effective amount of such a compound is that amount that produces a blood concentration of the compositions in a patient.

Patients that can be treated with the a compound described in this application, and the pharmaceutically acceptable salts, prodrugs, enantiomers and metabolites of such compounds, according to the methods of this invention include, for example, patients that have been diagnosed as having lung cancer, bone cancer, pancreatic cancer, skin cancer, cancer of the head and neck, cutaneous or intraocular melanoma, uterine cancer, ovarian cancer, rectal cancer or cancer of the anal region, stomach cancer, colon cancer, breast cancer, gynecologic tumors (e.g., uterine sarcomas, carcinoma of the fallopian tubes, carcinoma of the endometrium, carcinoma of the cervix, carcinoma of the vagina or carcinoma of the vulva), Hodgkin's disease, cancer of the esophagus, cancer of the small intestine, cancer of the endocrine system (e.g., cancer of the thyroid, parathyroid or adrenal glands), sarcomas of soft tissues, cancer of the urethra, cancer of the penis, prostate cancer, chronic or acute leukemia, solid tumors of childhood, lymphocytic lymphomas, cancer of the bladder, cancer of the kidney or ureter (e.g., renal cell carcinoma, carcinoma of the renal pelvis), or neoplasms of the central nervous system (e.g., primary CNS lymphoma, spinal axis tumors, brain stem gliomas or pituitary adenomas).

In further aspects of the present invention, the compositions described herein may be used to treat hematological malignancies including adult and pediatric AML, CML, ALL, CLL, myelodysplastic syndromes (MDS), myeloproliferative syndromes (MPS), secondary leukemia, multiple myeloma, Hodgkin's lymphoma and Non-Hodgkin's lymphomas.

Within such methods, pharmaceutical compositions are typically administered to a patient. As used herein, a “patient” refers to any warm-blooded animal, preferably a human. A patient may or may not be afflicted with a hematological malignancy. Accordingly, the above pharmaceutical compositions may be used to prevent the development of a malignancy, or delay it s appearance or reappearance, or to treat a patient afflicted with a malignancy. A hematological malignancy may be diagnosed using criteria generally accepted in the art. Pharmaceutical compositions may be administered either prior to or following surgical removal of primary tumors and/or treatment such as administration of radiotherapy or conventional chemotherapeutic drugs, or bone marrow transplantation (autologous, allogeneic or syngeneic).

The compositions provided herein may be used alone or in combination with conventional therapeutic regimens such as surgery, irradiation, chemotherapy and/or bone marrow transplantation (autologous, syngeneic, allogeneic or unrelated).

Kits for administering the compounds may be prepared containing a composition or formulation of the compound in question, or an enantiomer, prodrug, metabolite, or pharmaceutically acceptable salt of any of these, together with the customary items for administering the therapeutic ingredient.

When IMPDH inhibitors are used to treat cancer in combination with an agent that inhibits a cellular process regulated by GTP or ATP; including an α-tubulin polymerization inhibitor, an inhibitor of purine biosynthesis, an inhibitor of a receptor tyrosine kinase, or an antagonist of a GPCR; the compounds within the combination product can be administered substantially simultaneously or sequentially. If administered sequentially, the administration the IMPDH inhibitor is preferably administered before administration of the other compound. In a preferred embodiment the IMPDH inhibitor is given in a dosage sufficient to lower the GTP levels in target cells by 50%, or even more than 50%. If necessary, the IMPDH inhibitor can be administered repeatedly over a prolonged period of time. The IMPDH inhibitor can be administered at least 1, 2, 4, 6, 8, 12, 16, 20, 24, 30, 36, 40, 44, or 48 hours before administration of an agent that inhibits a cellular process regulated by GTP or ATP. In addition, the IMPDH inhibitor can be administered simultaneously with the agent that inhibits a cellular process regulated by GTP or ATP. In some instances it can be advantageous to administer the IMPDH inhibitor after the agent that inhibits a cellular process regulated by GTP or ATP.

Prodrugs of Mizoribine and its Aglycone

In another aspect, the present invention provides prodrugs of mizoribine, its aglycone and their analogues. The structure of the prodrugs is set forth in Formula I, below:

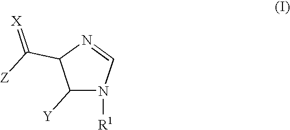

wherein, the symbol R¹ represents H, substituted or unsubstituted alkyl, substituted or unsubstituted heteroalkyl or saccharyl moieties. The symbol X represents O, S or NR′, in which R²is a member selected from H, substituted or unsubstituted alkyl, substituted or unsubstituted heteroalkyl, OH and NH². The symbol Y represents OR³ or NHR³, in which R³is a member selected from H, substituted or unsubstituted alkyl, substituted or unsubstituted heteroalkyl, acyl and P(O)OR¹²R¹³. R¹² and R¹³ are members independently selected from H, substituted or unsubstituted alkyl, substituted or unsubstituted heteroalkyl, acyl, acyloxyalkyl, and a single bond to an oxygen of the saccharyl of R¹. The symbol Z represents NR⁴R⁵, OR⁴ and SR⁴, in which R⁴ represents H, substituted or unsubstituted alkyl, substituted or unsubstituted heteroalkyl, a single bond to R³ or acyl; and R⁵ represents H, substituted or unsubstituted alkyl, substituted or unsubstituted heteroalkyl, acyl, acyloxycarbonyl, amino acid, peptidyl or acyloxyalkyl moieties.

In the compounds according to Formula I, the moieties represented by R³ and R⁴, together with the atoms to which they are attached, are optionally joined to form a 6-membered heterocycloalkyl ring. Moreover, when R³ is P(O)OR¹²R¹³, and R¹ is a saccharyl moiety, R¹³ and the saccharyl moiety and the atoms to which they are attached are optionally joined to form an 8-membered heterocycloalkyl ring. The compounds of the invention include at least one of the above-referenced 6-membered or 8-membered heterocycloalkyl ring systems.

In an exemplary embodiment, the invention provides a compound according to Formula II:

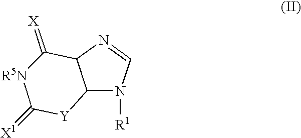

in which, the identity of X¹ is generally the same as is described for X, above. In a preferred embodiment, X¹ is O or S.

In another exemplary embodiment, the compounds of the invention have a structure according to Formula III:

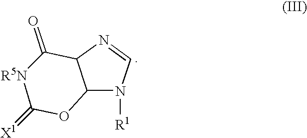

In yet another exemplary embodiment, the invention provides compounds that are glycones, having the structure according to Formula IV:

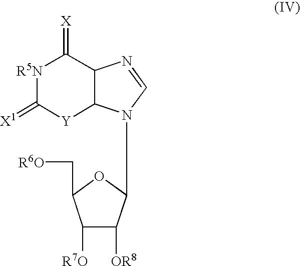

wherein R⁶, R⁷ and R⁸ are members independently selected from H, substituted or unsubstituted alkyl, substituted or unsubstituted heteroalkyl and acyl moieties. In one exemplary embodiment, one or more of R⁶, R⁷ and R⁸ is a protecting group. The protecting group prevents the oxygen atom to which it is attached from participating in, or interfering with, the reactions necessary to prepare the heterocyclic ring structure attached to the saccharyl moiety. Those of skill in the art will understand how to protect a particular functional group from interfering with a chosen set of reaction conditions. For examples of useful protecting groups, See Greene et al., PROTECTIVE GROUPS IN ORGANIC SYNTHESIS, John Wiley & Sons, New York, 1991.

In yet another embodiment, the compounds of the invention include a saccharyl moiety having free hydroxyl groups, having a structure according to Formula V:

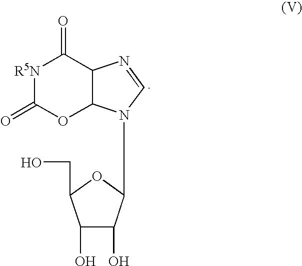

In each of the embodiments discussed herein, the R⁵ moiety may be H or a group that is cleaved by in vivo processes. Exemplary cleaveable groups include, but are not limited to, the structure according to Formula VI:

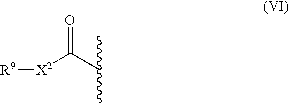

wherein X² is a member selected from O, CHR¹⁰R¹¹, and OC(O). The symbols R¹⁰ and R¹¹ independently represent H, substituted or unsubstituted alkyl, substituted or unsubstituted heteroalkyl, NH₂, NH₃⁺, COOH, COO⁻, OH, or SH. R⁹ is a member selected from H, substituted or unsubstituted alkyl, and substituted or unsubstituted heteroalkyl.

In a still further exemplary embodiment, the invention provides cyclic phosphodiester prodrugs of mizoribine, having a structure according to Formula VII:

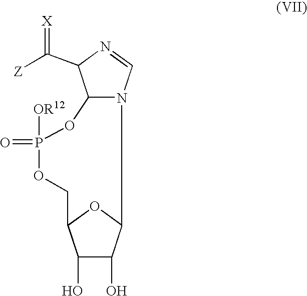

in which R¹², X and Y are substantially as described above.

Synthesis of Prodrugs of Mizoribine, its Aglycone and Their Analogues

The compounds of the invention are prepared and characterized by methods readily available to and understood by those of skill in the art. For example, a method of preparing the aglycone cyclic urethane derivatives according to Formula I is set forth in Scheme 1:

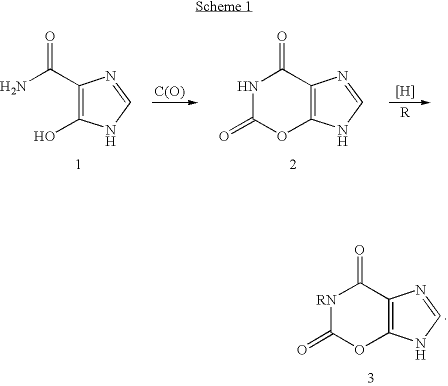

In Scheme I, the aglycone 1 is first contacted with an agent that donates a C(O) moiety, thereby closing the cyclic urethane ring, producing compound 2. Appropriate reagents for closing the ring include, but are not limited to, ethylchloroformate and 1,1′-carbonyldiimidazole. General methods of forming cyclic urethanes are known in the art. See, for example, Karagiri et al., J. Chem. Soc. Perkin Trans. I: 553 (1984); and Zou et al., J. Med. Chem. 34: 1951 (1991). Other useful reagents and methods for forming the cyclic urethane will be apparent to those of skill in the art.

In the optional second step of Scheme 1, the nitrogen of the six-membered ring is derivatized using a reagent capable of donating an “R” group, producing compound 3. Generally, prior to forming the linkage between the “R” group and the nitrogen of the bicyclic system, a chemical functionality on one of the reaction components is activated. One skilled in the art will appreciate that a variety of chemical functionalities, including hydroxy, amino, and carboxy groups, can be activated using a variety of standard methods and conditions. For example, a hydroxyl group of the cytotoxin or targeting agent can be activated through treatment with phosgene to form the corresponding chloroformate, or p-nitrophenylchloroformate to form the corresponding carbonate.

In an exemplary embodiment, the hydrogen is replaced with a “R” group using a nucleophilic substitution of an active species containing R, e.g., RX. The active species typically includes a leaving group, “X” in addition to the “R” group, which is to be appended to the nitrogen. Useful leaving groups include, but are not limited to, halides, azides, sulfonic esters (e.g., alkylsulfonyl, arylsulfonyl), oxonium ions, alkyl perchlorates, ammonioalkanesulfonate esters, alkylfluorosulfonates and fluorinated compounds (e.g., triflates, nonaflates, tresylates) and the like. The choice of these and other leaving groups appropriate for a particular set of reaction conditions is within the abilities of those of skill in the art (see, for example, March J, ADVANCED ORGANIC CHEMISTRY, 2nd Edition, John Wiley and Sons, 1992; Sandler S R, Karo W, ORGANIC FUNCTIONAL GROUP PREPARATIONS, 2nd Edition, Academic Press, Inc., 1983; and Wade L G, COMPENDIUM OF ORGANIC SYNTHETIC METHODS, John Wiley and Sons, 1980).

Once the “R” group is conjugated to the bicyclic structure, the “R” group is optionally further modified to produce a desired structure. Currently favored classes of reactions available for elaborating R⁵ of the prodrugs of the invention are those which proceed under relatively mild conditions. These include, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982.

Exemplary reaction types include the reaction of carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters. Hydroxyl groups can be converted to esters, ethers, aldehydes, etc. Haloalkyl groups are converted to new species by reaction with, for example, an amine, a carboxylate anion, thiol anion, carbanion, or an alkoxide ion. Dienophile (e.g., maleimide) groups participate in Diels-Alder. Aldehyde or ketone groups can be converted to imines, hydrazones, semicarbazones or oximes, or via such mechanisms as Grignard addition or alkyllithium addition. Sulfonyl halides react readily with amines, for example, to form sulfonamides. Amine or sulfhydryl groups are, for example, acylated, alkylated or oxidized. Alkenes, can be converted to an array of new species using cycloadditions, acylation, Michael addition, etc. Epoxides react readily with amines and hydroxyl compounds.

One skilled in the art will readily appreciate that many of these linkages may be produced in a variety of ways and using a variety of conditions. For the preparation of esters, see, e.g., March supra at 1157; for thioesters, see, March, supra at 362-363, 491, 720-722, 829, 941, and 1172; for carbonates, see, March, supra at 346-347; for carbamates, see, March, supra at 1156-57; for amides, see, March supra at 1152; for ureas and thioureas, see, March supra at 1174; for acetals and ketals, see, Greene et al. supra 178-210 and March supra at 1146; for acyloxyalkyl derivatives, see, PRODRUGS: TOPICAL AND OCULAR DRUG DELIVERY, K. B. Sloan, ed., Marcel Dekker, Inc., New York, 1992; for enol esters, see, March supra at 1160; for N-sulfonylimidates, see, Bundgaard et al., J. Med. Chem., 31:2066 (1988); for anhydrides, see, March supra at 355-56, 636-37, 990-91, and 1154; for N-acylamides, see, March supra at 379; for N-Mannich bases, see, March supra at 800-02, and 828; for hydroxymethyl ketone esters, see, Petracek et al. Annals NY Acad. Sci., 507:353-54 (1987); for disulfides, see, March supra at 1160; and for phosphonate esters and phosphonamidates.

The cyclic urethane of mizoribine and its analogues is formed using a methodology similar to that set forth above. In an exemplary embodiment, one or more of the mizoribine saccharyl hydroxide groups is protected prior to closing the urethane ring. An example of the reaction pathway utilizing a saccharyl precursor is set forth in

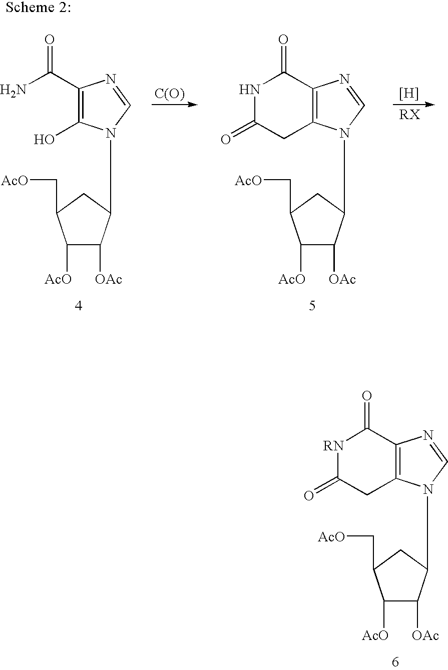

Scheme 2

In Scheme 2, the hydroxyl moieties of the saccharyl group of the starting material 4 are protected by the acetyl group, forming compound 5. Other appropriate protecting groups are readily available to those of skill in the art. The six-membered cyclic urethane is formed, and the nitrogen atom of the ring is optionally derivatized with an “R” group, forming compound 6. The saccharyl moiety is optionally deprotected.

The cyclic phosphate diester derivatives of the invention are prepared as set forth in Scheme 3:

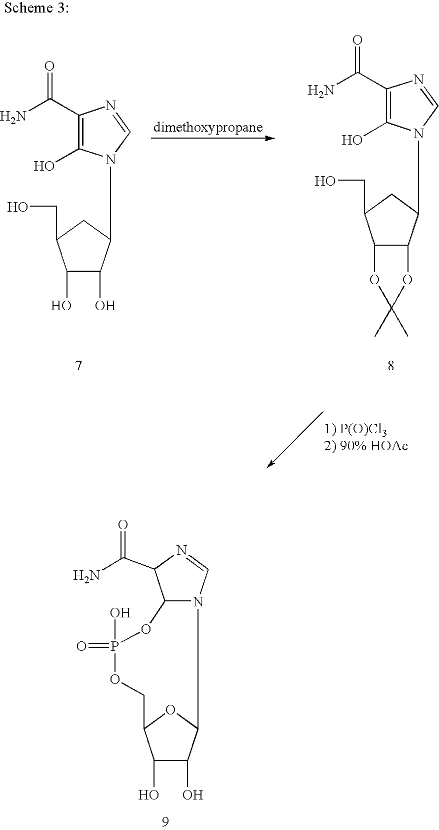

In Scheme 3, the vic-diol of the saccharyl moiety 7 is protected as the cyclic ketal 8. The protected derivative is phosphorylated and the phosphodiester ring is closed,. Following ring closure, the ketal protecting group is removed forming compound 9.

Immunosuppression Using Prodrugs of Mizoribine, Its Aglycone and Their Analogues

The above-described prodrugs of mizoribine and mizoribine aglycone can be used to provide immunosuppressive treatment to patients in need of such treatment.

The synthesis of guanosine nucleotides, and thus the activity of IMPDH, is particularly important in B and T-lymphocytes. See e.g., A. C. Allison et. al., Lancet II, 1179, (1975) and A. C. Allison et. al., Ciba Found. Symp., 48, 207, (1977). Thus, IMPDH is an attractive target for suppressing the immune system.

Inhibitors of IMPDH are known. Some inhibitors of IMPDH have been used as immunosuppressants, including mycophenolic acid and its prodrug mycophenylate mofetil. See e.g., R. E. Morris, Kidney Intl., 49, Suppl. 53:S-26, (1996); L. M. Shaw, et. al., Therapeutic Drug Monitoring, 17:690-699, (1995); and H. W. Sollinger, Transplantation, 60:225-232 (1995). However, use of both of these compounds is limited because of undesirable pharmacological properties, such as gastrointestinal toxicity and poor bioavailability. See e.g., L. M. Shaw, et. al., Therapeutic Drug Monitoring, 17:690-699, (1995) and A. C., Allison and E. M. Eugui, Immunological Reviews, 136:5-28 (1993). In addition, mizoribine has been used as an immunosuppressive agent in Japan. See e.g., Ishikawa, H. Curr. Med. Chem. 6:575-597 (1999).

Some patients are in need of immunosuppressive treatment to prevent rejection of a transplanted organ. Transplanted organs can include kidney, liver, heart, pancreas, bone-marrow and heart-lung transplants.

Other examples of immune system conditions or disorders which could benefit from treatment with an immunosuppressant agent include contact dermatitis; graft-vs-host disease in which donor immunological cells present in the graft attack host tissues in the recipient of the graft; diseases with proven or possible autoimmune components, such as rheumatoid arthritis, psoriasis, autoimmune uveitis, multiple sclerosis, allergic encephalomyelitis, systemic lupus erythematosis, aplastic anemia, pure red cell anemia, idiopathic thrombocytopenia, scleroderma, chronic active hepatitis, myasthenia gravis, Crohn's disease, ulcerative colitis, Graves-ophthalmopathy, sarcoidosis, primary biliary cirrhosis, primary juvenile diabetes, uveitis posterior, and interstitial lung fibrosis.

The following examples are provided to illustrate the present invention, but not to limit the claimed invention.

### EXAMPLES

### Example 1

Chronic Lymphocytic Leukemia (CLL) Cells Have a High Rate of α-Tubulin Polymerization and Depolymerization.

Lymphocyte Cultures. The Institutional Review Board of the University of California, San Diego approved this study, and all patients gave informed written consent to participation. Subjects had CLL according to National Cancer Institute (NCI) criteria of any Rai stage. None of the patients were in active chemotherapeutic treatments. Flow cytometric analysis determined that all specimens contained more than 90% CD5+CD19+B cells.

Peripheral blood from CLL patients or normal donors was layered on top of Ficoll-Paque Plus (Pharmacia, NJ) and centrifuged at 1200×g for twenty minutes. The enriched peripheral blood mononuclear cells (PBL) were washed several times with Ca++, Mg++-free HBSS. Normal B cells were purified from Buffy Coats using a RosetteSep Human B cell kit (StemCell Technologies Inc., Vancouver, Canada) according to the manufacturers' suggestions.

All primary cells were cultured in RPMI 1640 medium with 20% fetal bovine serum (regular medium) at a density of 2-5×10⁶ cell/ml. In some cases, the PBL were stimulated for 24 hours with 1 μg/ml anti-CD3 and anti-CD28 antibodies (Alexis, San Diego, Calif.) prior to immunoblot analysis.

α-tubulin Polymerization Assay. For each data point, 2×10⁶ of cells were incubated in 2 ml of regular medium containing drugs for various periods of time. The cells were then washed twice at room temperature, and resuspended in 50 μl of α-tubulin extraction buffer (1 mM MgC2, 2 m MEGTA, 0.5% NP-40, 20 mM Tris-HCl, pH=6.8) supplemented with 2 mM phenylmethysulfonyl fluoride, and a protease inhibitor cocktail (Sigma). After a brief but vigorous vortex, the lysates were incubated at room temperature for 5 minutes, and then centrifuged at 16,000×g for 5 minutes to separate the soluble from polymerized α-tubulin. The supernatant and pellet fractions were resolved on 10-20% pre-cast tris-glycine gels (Novex, San Diego, Calif.) and then subjected to immune blotting with a specific anti-α-tubulin antibody.

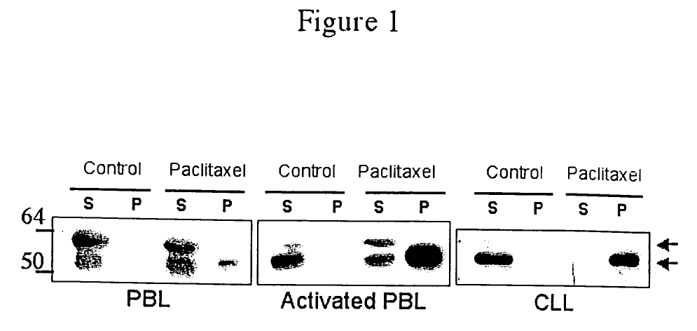

Microtubule polymerization dynamics in PBL and CLL. The cytosolic (S) monomeric and particulate-bound (P) polymerized forms of α-tubulin were separated by centrifugation from drug-treated cells and assayed by immunoblotting. In normal PBL (FIG. 1, left panel), α-tubulin was found mostly in the soluble fraction, with an apparent molecular weight of 61 kDa. Treatment with the microtubule-polymerizing agent paclitaxel for one hour induced only a minimal increase of α-tubulin in the particulate fraction. In PBL activated for 24 hrs in the presence of anti-CD3 and anti-CD28 antibodies (FIG. 1, middle panel) the majority of α-tubulin was still in the soluble fraction, but had a diminished apparent molecular weight of 54 kDa (lower band). In contrast to the results with resting PBL, when the activated PBL were treated with paclitaxel for one hour, most of the α-tubulin shifted to the particulate fraction.

CLL cells (FIG. 1, right panel) expressed almost exclusively the 54 kDa α-tubulin band in the soluble subcellular fraction. Treatment with paclitaxel induced the nearly complete relocalization of α-tubulin to the particulate fraction. Thus, although the CLL cells were slow growing, they exhibited a rapid rate of α-tubulin turnover.

### Example 2

Mizoribine and Indanocine are Synergistically Toxic to CLL Cells.

Indanocine is commercially available from Calbiochem (Cat. No. 402080). Leoni L. M., et al., J Natl Cancer Inst. 92:217-224 (2000). Hua X. H., et al., Cancer Res. 61:7248-7254 (2001). CLL cells were isolated and cultured as described above. CLL cells at a density of 1×10⁶ ml were incubated for 24 hours in RPMI 1640 medium supplemented with 10% fetal bovine serum in the presence of 1 μM indanocine (abbreviated 178) and either 0, 1, or 10 μM mizoribine. Cell viability was assessed by the MTT [3-(4,5-dimethylthiazol-2-yl)-2,5-diphenyltetrazolium bromide) dye assay.

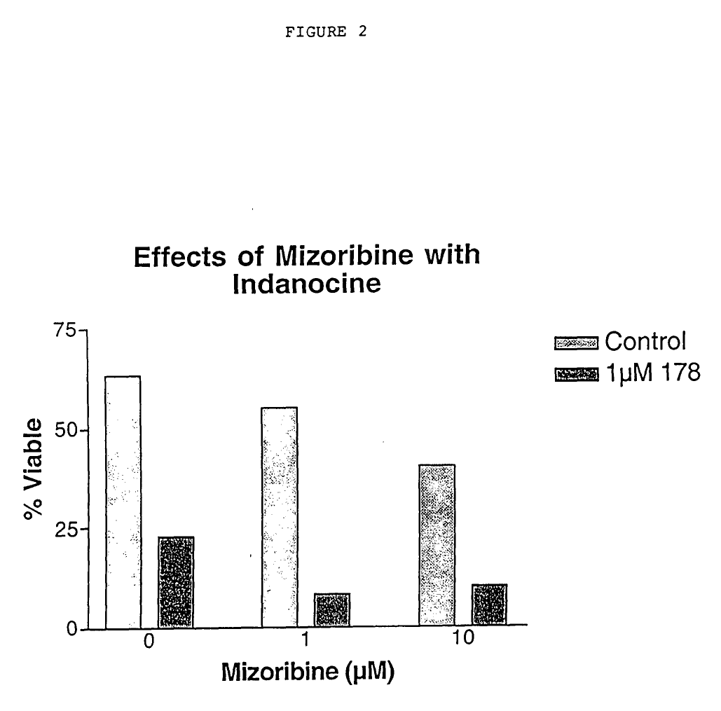

In the absence of drugs approximately 65% of the CLL cells were viable. (FIG. 2) Addition of 1 μM indanocine resulted in death of more than half of the CLL cells (24% viability). Addition of 1 or 10 μM mizoribine alone resulted in negligible decrease in viability (55% and 45% respectively). The combination of mizoribine and indanocine resulted in a surprisingly large decrease in CLL cell viability. At both concentrations of mizoribine tested, cell viability dropped below 10% in the presence of indanocine. The effect was especially pronounced at 1 μM mizoribine.

### Example 3

Mizoribine is Toxic to MTAP-Deficient Cells

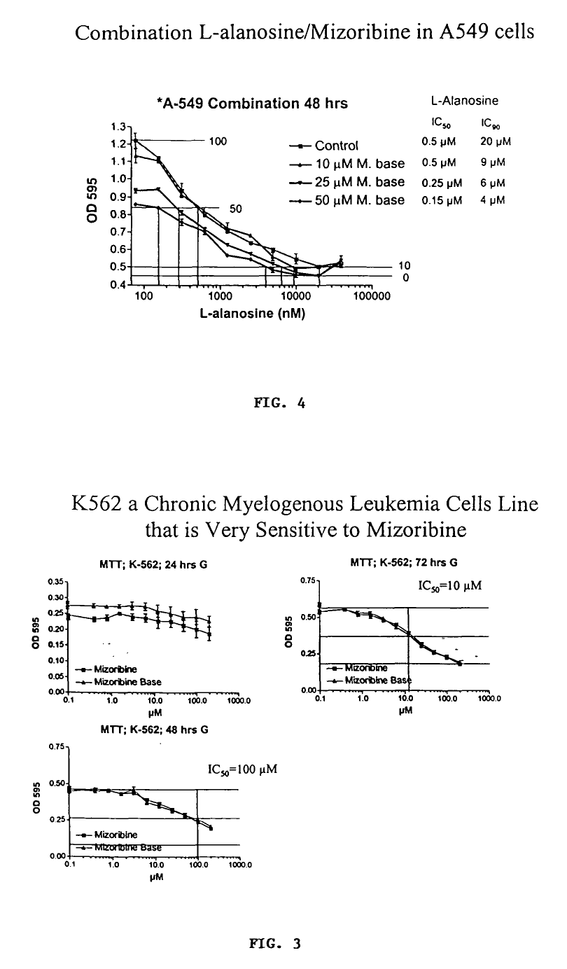

MTAP-deleted chronic myelogenous leukemia cells (K562) were pre-treated for the indicated times (24, 48, 72 hours) with concentrations of mizoribine (squares) or mizoribine base (triangles) from 200 μM to 0.5 μM. At the end of the incubation time cell proliferation was tested by the MTT dye assay. As indicated by FIG. 3, the IC₅₀ (amount of drug needed to block proliferation by 50%) of mizoribine or its base progressively declines as the time of exposure to drug increases. After 48 hours of incubation with mizoribine or mizoribine base, the IC₅₀ for the drug is approximately 100 micromolar. After 72 hours of incubation with mizoribine or mizoribine base, the IC₅₀ for the drug is approximately 10 micromolar.

### Example 4

Mizoribine, in Combination with L-Alanosine is Synergistically Toxic to MTAP-Deficient Cells

MTAP-deleted lung cancer cells (A549) were pre-treated for 24 hours with control vehicle (square), or the indicated concentrations of mizoribine-base (10 μM, 25 μM and 50 μM). After 24 hours in culture L-alanosine was added at decreasing concentrations (½ dilutions) starting at 40 μM. Cell were then incubated for an additional 48 hours. Cell proliferation was tested by the MTT assay. As indicated by FIG. 4, the IC₅₀ and IC₉₀ (amount of drug needed to block proliferation by 50% or 90%) of L-alanosine declines in the presence of mizoribine base.

Without mizoribine base the IC₅₀ of L-alanosine is 0.5 micromolar and the IC₉₀ is 20 micromolar. In the presence of 10 micromolar mizoribine base the IC₅₀ of L-alanosine is 0.5 micromolar and the IC₉₀ is 9 micromolar. In the presence of 25 micromolar mizoribine base the IC₅₀ of L-alanosine is 0.25 micromolar and the IC₉₀ is 6 micromolar. In the presence of 50 micromolar mizoribine base the IC₅₀ of L-alanosine is 0.15 micromolar and the IC₉₀ is 4 micromolar.

The disclosure of a co-pending application, Methods for Inhibiting Protein Kinases in Cancer Cells, Attorney Docket No. 02307O-126810US, also filed on Aug. 1, 2003 is herein incorporated by reference, as is its priority document, U.S. Provisional Application No. 60/400,568, filed Aug. 2, 2002.

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

What is claimed is:

1. A method for treating cancer comprising administering to a subject in need of such treatment a therapeutically effective amount of
(a) a member selected from an inhibitor of inosine monophosphate dehydrogenase (IMPDH), an enantiomer of such a compound, a prodrug of such a compound, a pharmaceutically acceptable salt of such a compound, and combinations thereof; and
(b) an agent that inhibits a cellular process regulated by GTP or ATP.

2. The method of claim 1, wherein the agent that inhibits a cellular process regulated by GTP is selected from the group consisting of an inhibitor of α-tubulin polymerization, a prodrug therefor, a pharmaceutically acceptable salt thereof, and combinations thereof.

3. The method of claim 2, wherein the IMPDH inhibitor is selected from the group consisting of mizoribine, mizoribine aglycone, mycophenolate mofetil, tiazofurin, viramidine, and ribivarin.

4. The method of claim 2, wherein the α-tubulin polymerization inhibitor is selected from the group consisting of indanocine, indanrorine, vincristine, vinblastine, vinorelbine, combretastatin-A, and colchicine.

5. The method of claim 2, wherein the IMPDH inhibitor is mizoribine and the α-tubulin polymerization inhibitor is indanocine.

6. The method of claim 2, wherein the cancer is a slow growing cancer.

7. The method of claim 6, wherein the slow growing cancer has a high rate of α-tubulin turnover.

8. The method of claim 6, wherein the slow growing cancer is selected from the group consisting of chronic lymphocytic leukemia, chronic myelogenous leukemia, non-Hodgkins lymphoma, multiple myeloma, chronic granulocytic leukemia, cutaneous T cell lymphoma, low grade lymphomas, slow growing breast cancer, slow growing prostate cancer, and slow growing thyroid cancer.

9. A composition for treating cancer in a subject in need of such treatment comprising therapeutically effective amounts of
(a) a member selected from an inhibitor of inosine monophosphate dehydrogenase (IMPDH), an enantiomer of such a compound, a prodrug of such a compound, a pharmaceutically acceptable salt of such a compound, and combinations thereof; and
(b) an agent that inhibits a cellular process regulated by GTP or ATP.

10. The composition of claim 9, wherein the agent that inhibits a cellular process regulated by GTP is a member selected from an inhibitor of α-tubulin polymerization, a prodrug therefor, or a pharmaceutically acceptable salt thereof, and combinations thereof.

11. The composition of claim 10, wherein the IMPDH inhibitor is selected from the group consisting of mizoribine, mizoribine aglycone, mycophenolate mofetil, tiazofurin, viramidine, and ribivarin.

12. The composition of claim 10, wherein the α-tubulin polymerization inhibitor is selected from the group consisting of indanocine, vincristine, vinblastine, vinorelbine, combretastatin-A, and colchicine.

13. The composition of claim 10, wherein the IMPDH inhibitor is mizoribine and the α-tubulin polymerization inhibitor is indanocine.

14. The method of claim 1, wherein the agent that inhibits a cellular process regulated by GTP is a member selected from a precursor of 9-beta-D-arabinofuranosylguanine 5′-triphosphate (Ara-GTP), a prodrug therefore, a pharmaceutically acceptable salt thereof, and combinations thereof.

15. The method of claim 14, wherein the IMPDH inhibitor is selected from the group consisting of mizoribine, mizoribine aglycone, mycophenolate mofetil, tiazofurin, viramidine, and ribivarin.

16. The method of claim 14, wherein the precursor of Ara-GTP is selected from the group consisting of guanine arabinoside (Ara-G) and Nelarabine.

17. The method of claim 14, wherein the cancer is a lymphoma or a leukemia.

18. The composition of claim 9, wherein the agent that inhibits a cellular process regulated by GTP is a member selected from a precursor of Ara-GTP, a prodrug therefor, or a pharmaceutically acceptable salt thereof, and combinations thereof.

19. The composition of claim 18, wherein the IMPDH inhibitor is selected from the group consisting of mizoribine, mizoribine aglycone, mycophenolate mofetil, tiazofurin, viramidine, and ribivarin.

20. The composition of claim 18, wherein the precursor of Ara-GTP is selected from the group consisting of guanine arabinoside (Ara-G) and Nelarabine.

21. The method of claim 1, wherein the agent that inhibits a cellular process regulated by GTP is a member selected from an inhibitor of the de novo pathway of purine biosynthesis, a prodrug therefor, or a pharmaceutically acceptable salt thereof, and combinations thereof.

22. The method of claim 21, wherein the IMPDH inhibitor is selected from the group consisting of mizoribine, mizoribine aglycone, mycophenolate mofetil, tiazoflirin, viramidine, and ribivarin.

23. The method of claim 21, wherein the IMPDH inhibitor is mizoribine.

24. The method of claim 21, wherein the IMPDH inhibitor is mizoribine aglycone.

25. The method of claim 21, wherein the inhibitor of the de novo pathway of purine biosynthesis is selected from the group consisting of L-alanosine, methotrexate, trimetrexate, 10-propargyl-5,8-dideazafolic acid (PDDF), N-[5-[N-(3,4-dihydro-2-methyl-4-oxoquinazolin-6-ylmethyl)-N-methylamino]-2-thenoyl]-L-glutamic acid (ZD1694, Tomudex), N-[4-[2-(2-amino-3,4-dihydro-4-oxo-7H-pyrrolo[2,3-d]-pyrimidin-5-yl)ethyl]-benzoyl]-L-glutamic acid (LY231514), 6-(2′-formyl-2′naphthyl-ethyl)-2-amino-4(3H)-oxoquinazoline (LL95509), (6R,S)-5,10-dideazatetrahydrofolic acid (DDATHF), 4-[2-(2-amino-4-oxo-4,6,7,8-tetrahydro-3Hpyrimidino[5,4,6][1,4]-thiazin-6yl)-(S)-ethyl]-2,5-thienoylamino-L-glutamic acid (AG2034), and N-[5-(2-[(2,6-diamino-4(3H)-oxopyrimidin-5-yl)thio]ethyl)thieno-2-yl]-L-glutamic acid (AG2009).

26. The method of claim 21, wherein the cancer comprises a population of cells deficient in the enzyme methyladenosine phosphorylase (MTAP).

27. A method for treating cancer in a subject in need of such treatment, wherein the cancer comprises of a population of cells deficient in the enzyme methlyadenosine phosphorylase (MTAP), comprising:
administering to the subject a therapeutically effective amount of a member selected from an inhibitor of inosine monophosphate dehydrogenase (IMPDH), an enantiomer of such a compound, a prodrug of such a compound, a pharmaceutically acceptable salt of such a compound, and combinations thereof.

28. The method of claim 27, wherein the IMPDH inhibitor is selected from the group consisting of mizoribine, mizoribine aglycone, mycophenolate mofetil, tiazofurin, viramidine, and ribivarin.

29. The method of claim 27, wherein the IMPDH inhibitor is mizoribine.

30. The method of claim 27, wherein the IMPDH inhibitor is mizoribine aglycone.

31. The composition of claim 9, wherein the agent that inhibits a cellular process regulated by GTP is a member selected from an inhibitor of the de novo pathway of purine biosynthesis, a prodrug therefor, a pharmaceutically acceptable salt thereof, and combinations thereof.

32. The composition of claim 31, wherein the IMPDH inhibitor is selected from the group consisting of mizoribine, mizoribine aglycone, mycophenolate mofetil, tiazofurin, viramidine, and ribivarin.

33. The composition of claim 31, wherein the inhibitor of the de novo pathway of purine biosynthesis is selected from the group consisting of L-alanosine, methotrexate, trimetrexate, 10-propargyl-5,8-dideazafolic acid (PDDF), N-[5-[N-(3,4-dihydro-2-methyl-4-oxoquinazolin-6-ylmethyl)-N-methylamino]-2-thenoyl]-L-glutamic acid (ZD1694, Tomudex), N-[4-[2-(2-amino-3,4-dihydro-4-oxo-7H-pyrrolo[2,3-d]-pyrimidin-5-yl)ethyl]-benzoyl]-L-glutamic acid (LY231514), 6-(2 ′-formyl-2′naphthyl-ethyl)-2-amino-4(3H)-oxoquinazoline (LL95509), (6R,S)-5,10-dideazatetrahydrofolic acid (DDATHF), 4-[2-(2-amino-4-oxo-4,6,7,8-tetrahydro-3Hpyrimidino[5,4,6][1,4]-thiazin-6yl)-(S)-ethyl]-2,5-thienoylamino-L-glutamic acid (AG2034), and N-[5-(2-[(2,6-diamino-4(3H)-oxopyrimidin-5-yl)thio]ethyl)thieno-2-yl]-L-glutamic acid (AG2009).

34. The composition of claim 31, wherein the inhibitor of the de novo pathway of purine biosynthesis is L-alanosine.

35. The method of claim 1, wherein the agent that inhibits a cellular process regulated by GTP is an antagonist of a G-protein coupled receptor (GPCR).

36. The method of claim 35, wherein the IMPDH inhibitor is selected from the group consisting of mizoribine, mizoribine aglycone, mycophenolate mofetil, tiazofurin, viramidine, and ribivarin.

37. The method of claim 35, wherein the GPCR antagonist is selected from the group consisting of atrasentan, leuprolide, goserelin, and octreotide.

38. The method of claim 35, wherein the cancer is prostate cancer.

39. The composition of claim 9, wherein the agent that inhibits a cellular process regulated by GTP is a member selected from an antagonist of a G-protein coupled receptor (GPCR), a prodrug therefor, or a pharmaceutically acceptable salt thereof.

40. The composition of claim 39, wherein the IMPDH inhibitor is selected from the group consisting of mizoribine, mizoribine aglycone, mycophenolate mofetil, tiazofurin, viramidine, and ribivarin.

41. The composition of claim 39, wherein the GPCR antagonist is selected from the group consisting of atrasentan, leuprolide, goserelin, and octreotide.

42. A compound having the formula: 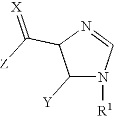
wherein
R¹ is a member selected from H, substituted or unsubstituted alkyl, substituted or unsubstituted heteroalkyl and saccharyl moieties;
X is a member selected from O, S and NR²
in which
R² is a member selected from H, substituted or unsubstituted alkyl, substituted or unsubstituted heteroalkyl, OH and NH₂;
Y is a member selected from OR³ and NHR³
in which
R³is a member selected from H, substituted or unsubstituted alkyl, substituted or unsubstituted heteroalkyl, acyl and P(O)OR¹²R¹³
wherein
R¹² and R¹³ are members independently selected from H, substituted or unsubstituted alkyl, substituted or unsubstituted heteroalkyl, acyl, acyloxyalkyl, and a single bond to an oxygen of said saccharyl of R¹;
Z is a member selected from NR⁴R⁵, OR⁴ and SR⁴
in which
R⁴is a member selected from H, substituted or unsubstituted alkyl, substituted or unsubstituted heteroalkyl, a single bond to R³ and acyl;
R⁵ is a member selected from H, substituted or unsubstituted alkyl, substituted or unsubstituted heteroalkyl, acyl, acyloxycarbonyl, amino acid, peptidyl and acyloxyalkyl moieties; and
R³ and R⁴, together with the atoms to which they are attached, are optionally joined to form a 6-membered heterocyclic ring;
when R³ is P(O)OR¹²R¹³, and R¹ is a saccharyl moiety, R¹³ and said saccharyl moiety and the atoms to which they are attached are optionally joined to form an 8-membered heterocyclic ring, with the proviso that said compound includes at least one of said 6-membered or said 8-membered heterocyclic ring system.

43. The compound according to claim 42, having the formula: 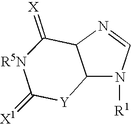
in which
X¹ is a member selected from O and S.

44. The compound according to claim 43, having the formula:

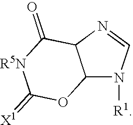

45. The compound according to claim 43 having the formula: 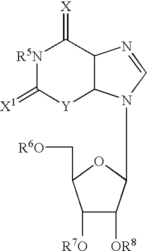
wherein
R⁶, R⁷ and R⁸ are members independently selected from H, substituted or unsubstituted alkyl, substituted or unsubstituted heteroalkyl and acyl moieties.

46. The compound according to claim 45 having the formula:

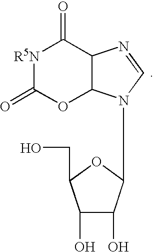

47. The compound according to claim 42, wherein R⁵ has the formula: 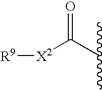
wherein
X² is a member selected from O, CHR¹⁰R¹¹, and OC(O)
wherein
R¹⁰ and R¹¹ are members independently selected from H, substituted or unsubstituted alkyl, substituted or unsubstituted heteroalkyl, NH₂, NH₃⁺, COOH, COO—, OH, and SH; and
R⁹ is a member selected from H, substituted or unsubstituted alkyl, and substituted or unsubstituted heteroalkyl.

48. The compound according to claim 42 having the formula:

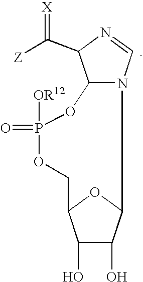

49. The compound according to claim 48, having the formula:

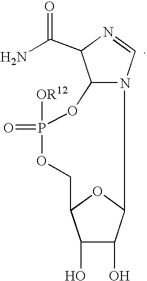

50. A pharmaceutical formulation comprising a compound according to claim 42 and a pharmaceutically acceptable carrier.

51. A method for treating cancer comprising administering to a subject in need of such treatment a compound selected from the group consisting of mizoribine, mizoribine aglycone, prodrugs of mizoribine, and prodrugs of mizoribine aglycone, wherein the compound is administered in an amount sufficient to maintain a plasma level of the compound of between 0.5 and 50 micromolar for between 6 and 72 hours.

52. The method of claim 51, wherein the plasma level of compound is between 1 and 30 micromolar for between 8 and 48 hours.

53. The method of claim 51, wherein the plasma level of compound is between 5 and 25 micromolar for between 10 and 24 hours.

54. The method of claim 51, wherein the plasma level of compound is at least 10 micromolar for at least 12 hours.

55. The method of claim 51, wherein the compound comprises a pharmaceutically acceptable carrier.

56. The method of claim 51, wherein the compound is administered parenterally.

57. The method of claim 51, wherein the compound is administered orally.

58. The method of claim 51, wherein the compound is described by the formula of claim 42.

59. A method of treating an immune system condition by providing an immunosupressive agent, the method comprising administering to a subject in need of such treatment a therapeutically effective amount of a compound described by the formula of claim 42.

60. The method of claim 59, wherein the compound comprises a pharmaceutical carrier.

61. The method of claim 59, wherein the immune system condition is rejection of a transplanted organ.

62. The method of claim 59, wherein the immune system condition is an autoimmune disease.
